(19) United States
(12) Patent Application Publication
(10) Pub. No.: US 2026/0232269 A1
(43) Pub. Date: Aug. 13, 2026

(54) ON-BODY SYSTEM AND INSERTION SYSTEM

(71) Applicant: PercuSense, Inc., Valencia, CA (US)

(72) Inventors: Katherine Wolfe, Dunwoody, GA (US); Bella Nava, Los Angeles, CA (US); Benjamin Pagliuso, Marina del Rey, CA (US)

(73) Assignee: PercuSense, Inc., Valencia, CA (US)

(21) Appl. No.: 19/258,739

(22) Filed: Jul. 2, 2025

Related U.S. Application Data
(60) Provisional application No. 63/667,192, filed on Jul. 3, 2024.
(60) Provisional application No. 63/693,332, filed on Sep. 11, 2024.

(51) Int. Cl. A61B 5/00; A61B 5/145

(52) U.S. Cl. CPC A61B 5/6849; A61B 5/14503; A61B 2560/063

(57) ABSTRACT

An on-body assembly is described. The on-body assembly includes an electronics module with a top case and a bottom case. The assembly further includes a base with a clip to retain the electronics module. The base also has an interior base that includes a sensor platform, a sensor channel formed between the sensor platform and a base aperture, and a retention tab located over the sensor channel. The assembly also includes a sensor with a proximal end, a mid-portion and a distal end. The proximal end is coupled to the sensor platform, the mid-portion is positioned within the sensor channel, and the distal end extends through the base aperture.

### RELATED APPLICATIONS

This application claims the benefit of U.S. provisional application No. 63/667,192 filed on Jul. 3, 2024. The application listed above is hereby incorporated by reference in its entireties for all purposes.

### BACKGROUND

The use of continuous glucose sensors to diagnose and treat conditions associated with diabetes has steadily increased. Since being made available as an over the counter item, continuous glucose sensors have additionally been used to help provide insight regarding general health for non-diabetic users as well. Moreover, there has been interest in measuring or detecting additional molecules or analytes of interest such as ketones, lactate and oxygen.

The analytes of interest are often found in interstitial fluid necessitating reliable placement of the sensor within interstitial fluid. Commonly, insertion tools or inserters utilize “needles” or “sharps” to place the sensor within the interstitial fluid of a subject. However, it is not uncommon for malfunctions to occur based on user error, improper training, or a host of other issues associated with either or both the sensor or the insertion tool.

Thus, a need exists for reliable sensor insertion devices, systems and methods, particularly for use in conjunction with percutaneous sensors, that are easy to use by the patient and less prone to malfunctions.

### SUMMARY

Described herein are methods, systems, and devices relating to the on-body assemblies, and insertion device assemblies to enable the storage, sterilization, and application of implantable sensors to acquire and interpret data from one or more biochemical analytes or markers in near real time so changes in the physiological state of a subject can be recognized.

Methods and structures disclosed herein enable sensors configured to detect the presence of at least one or more biochemical analytes to be implanted within a subject. The methods and structures include embodiments to enable sterilization of disposable components while also minimizing or reducing medical waste by enabling reuse of durable components.

For the purpose of summarizing the disclosure, certain aspects, advantages and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

FIG. 1A is an exemplary pseudo isometric illustration of an insertion device, in accordance with embodiments of the present invention.

FIG. 1B is an exemplary side view illustration of the insertion device that includes the reusable portion and the disposable portion.

FIG. 2A is an exemplary cross-section view of select components of the reusable portion in a first position, in accordance with embodiments of the present invention.

FIG. 2B is an exemplary cross-section view of the disposable portion in the first state, in accordance with embodiments of the present invention.

FIG. 3A is an exemplary illustration of a partial cross-section view of the reusable portion in the second position while the disposable portion is in the first state, in accordance with embodiments of the present invention.

FIG. 3B is an exemplary illustration of a cross-section view of select parts of the reusable portion in the second position and the disposable portion in the first state, in accordance with embodiments of the present invention.

FIG. 4A is an exemplary cross-section view of the reusable portion in the second position and the disposable portion in a second state, in accordance with embodiments of the present invention.

FIG. 4B is a pseudo-isometric cross-section view of the reusable portion in the second position and the disposable portion without the safety in the second state, in accordance with embodiments of the present invention.

FIG. 4C is an exemplary illustration of a cross-section of select components of the reusable portion in the second position, in accordance with embodiments of the present invention.

FIG. 5 is a cross-section view through the reusable portion in the first position illustrating interactions between the actuator, the housing, the tail, and the latch, in accordance with embodiments of the present invention.

FIG. 6A is an exemplary cross-section view of select parts of the reusable portion (without the insertion spring) in an intermediary position between the first position and the second position, and select parts of the disposable portion in a third state, in accordance with embodiments of the present invention.

FIG. 6B is an exemplary cross-section view of select portions of the reusable portion in the first position and select portions of the disposable portion in a fourth state, in accordance with embodiments of the present invention.

FIG. 6C is an exemplary pseudo-isometric cross-section illustration of select parts of the disposable components of the insertion device in an intermediate position, between the third state (FIG. 6A) and the fourth state (FIG. 6B), in accordance with embodiments of the present invention.

FIG. 7 is an exemplary cross-section view of portions of the reusable portion in the first position and portions of the disposable portion in a fifth state, in accordance with embodiments of the present invention.

FIG. 8A is an exemplary cross-section view of select components of the disposable portion being detached or separated from select portions of the reusable portion, in accordance with embodiments of the present invention.

FIG. 8B is an exemplary cross-section view of select components of the disposable portion interfaced with select components of the reusable portion, in accordance with embodiments of the present invention.

FIG. 9A is an exemplary pseudo-isometric view of an on-body assembly, in accordance with embodiments of the present invention.

FIG. 9B is a top view of the on-body assembly, in accordance with embodiments of the present invention.

FIG. 10A is a top view of the base, in accordance with embodiments of the present invention.

FIG. 10B is a pseudo-isometric view of the base, in accordance with embodiments of the present invention

FIG. 10C is an exemplary cross-section of the base that illustrates the aperture traversing through the base from the interior to an exterior, in accordance with embodiments of the present invention.

FIG. 10D is an exemplary pseudo-isometric cross-section of the base that illustrates how the portions of the sensor interact with the base to influence positioning of the distal end as it protrudes through the base aperture, in accordance with embodiments of the present invention.

FIG. 10E is an exemplary cross-section of the base that illustrates a bias face formed in the sensor channel that biases the sensor at an angle away from vertical (as illustrated), in accordance with embodiments of the present invention.

FIG. 11A is a pseudo-isometric cross-section view of the base, the patch and an adhesive, in accordance with embodiments of the present invention.

FIG. 11B is a cross-section view of the base illustrating the interaction between the bias face, the sensor channel and the retention face of the retention tab, in accordance with embodiments of the present invention.

FIGS. 12A-12C are exemplary pseudo-isometric cross-sections of the electronics module that also includes the base, patch, in accordance with embodiments of the present invention.

FIGS. 12D and 12E are cross-section views of the base, electronics module and patch in accordance with embodiments of the present invention.

FIG. 13A is a view of a bottom face of the bottom case of the electronics module, in accordance with embodiments of the present invention.

FIGS. 13B and 13C are pseudo-isometric cross-section views of the electronics module, in accordance with embodiments of the present invention.

FIG. 13D is a pseudo-isometric view of the electronics module in accordance with embodiments of the present invention.

FIG. 14A-14D are various illustrations of an alternative embodiment of select elements of the on body assembly that includes the electronics module mounted to the base in accordance with embodiments of the present invention.

FIG. 15 is an isometric view of a sensor assembly, in accordance with embodiments of the present invention.

FIG. 15A is an isometric view of select components of the sensor assembly, in accordance with embodiments of the present invention.

FIG. 15B is a cross-section view of select components of the sensor assembly in a first state, in accordance with embodiments of the present invention.

FIG. 16 is an isometric view of an insertion assembly in a first position, in accordance with embodiments of the present invention.

FIG. 16A is a cross-section view of the insertion assembly in a first position, in accordance with embodiments of the present invention.

FIG. 17 is an isometric view of positioning of the insertion assembly in a first position over select components of the sensor assembly in a first state, in accordance with embodiments of the present invention.

FIG. 18A is a side view of select components of the sensor assembly in a first state and the insertion assembly in a second position, in accordance with embodiments of the present invention.

FIG. 18B is a cross-section view of select components of the sensor assembly in a first state and the insertion assembly in a second position, in accordance with embodiments of the present invention.

FIG. 18C is a cross-section view of select components of the sensor assembly in a second state and the insertion assembly in a second position, in accordance with embodiments of the present invention.

FIG. 18D is a cross-section view of select components of the sensor assembly before a transition between the second state and a third state and the insertion assembly in an intermediate position between the second position and the first position, in accordance with embodiments of the present invention.

FIG. 18E is a cross-section view of select components of the sensor assembly in a fourth state and the insertion assembly in a first position, in accordance with embodiments of the present invention.

FIG. 18F is a cross-section view of select components of the sensor assembly between the third state and the fourth state and the insertion assembly in a first position, in accordance with embodiments of the present invention

FIG. 19 is a cross-section view of select components of the insertion assembly and the sensor assembly in a fourth state that includes separation of the base, in accordance with embodiments of the present invention.

FIG. 20A is a view of the bottom of select components of the sensor assembly and the insertion assembly illustrating interactions between the top and the slider, in accordance with embodiments of the present invention.

FIG. 20B is a view of the bottom of select components of the sensor assembly and the insertion assembly illustrating removal of sensor assembly from the insertion assembly, in accordance with embodiments of the present invention.

FIG. 20C is an isometric view illustrating separation of select components of the sensor assembly from the insertion assembly, in accordance with embodiments of the present invention.

FIG. 20D is an isometric view illustrating the insertion assembly in the first position after removal of the remaining components of the sensor assembly, in accordance with embodiments of the present invention.

### DETAILED DESCRIPTION

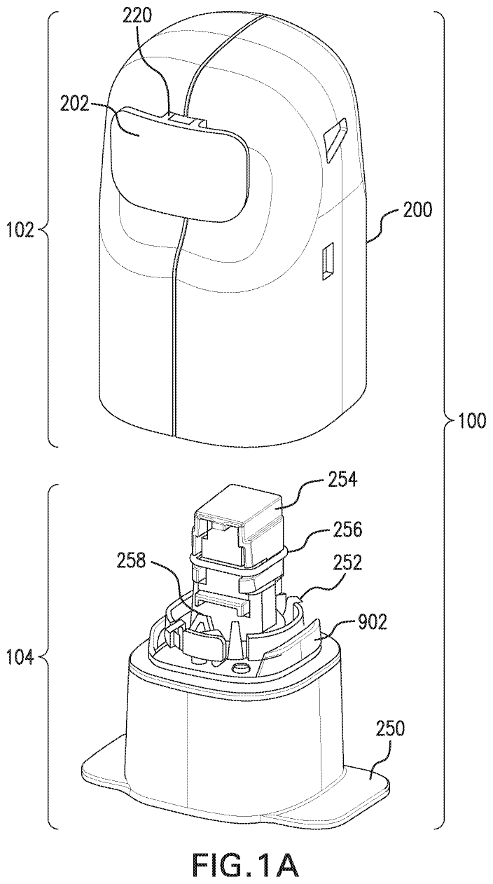

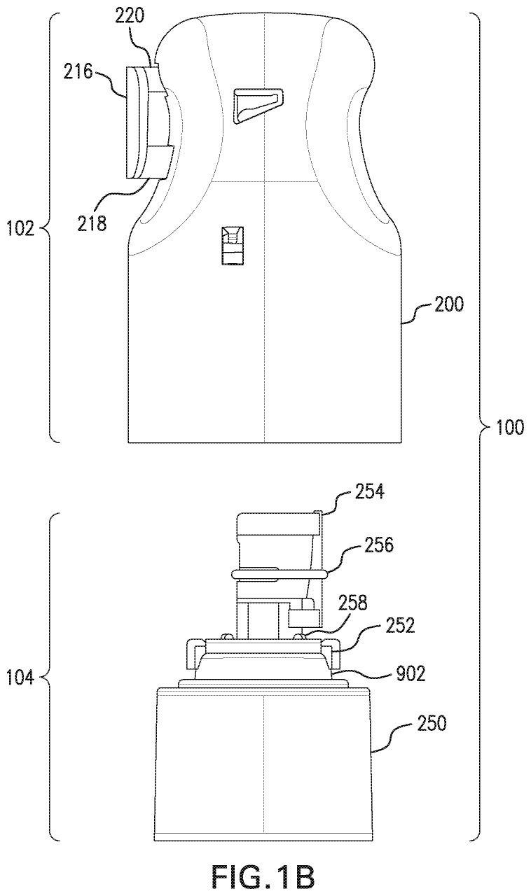

FIG. 1A is an exemplary pseudo isometric illustration of a partially exploded view of an insertion device 100, in accordance with embodiments of the present invention. The insertion device 100 includes the following two subassemblies; a reusable portion 102 and a disposable portion 104.

FIG. 1B is an exemplary side view illustration of the insertion device 100 that includes the reusable portion 102 and the disposable portion 104. The insertion device 100 can be used to insert a medical device such as a sensor into a subject. In preferred embodiments, the sensor can be inserted through the skin of the subject into the dermis or subcutaneous tissue of the subject. In still other embodiments, the insertion device 100 may be used to place a sensor or medical device within other tissue, organs or even the musculature of a subject.

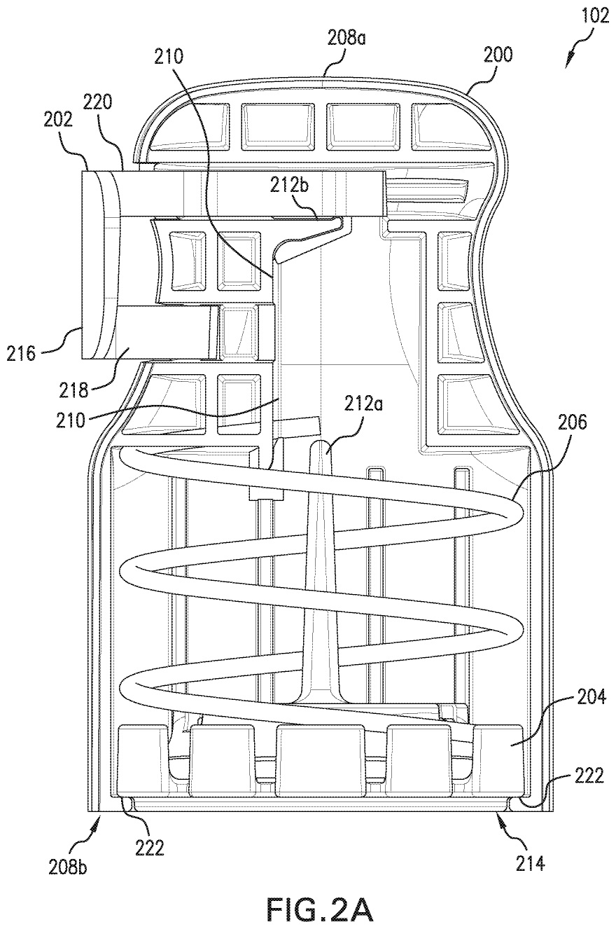

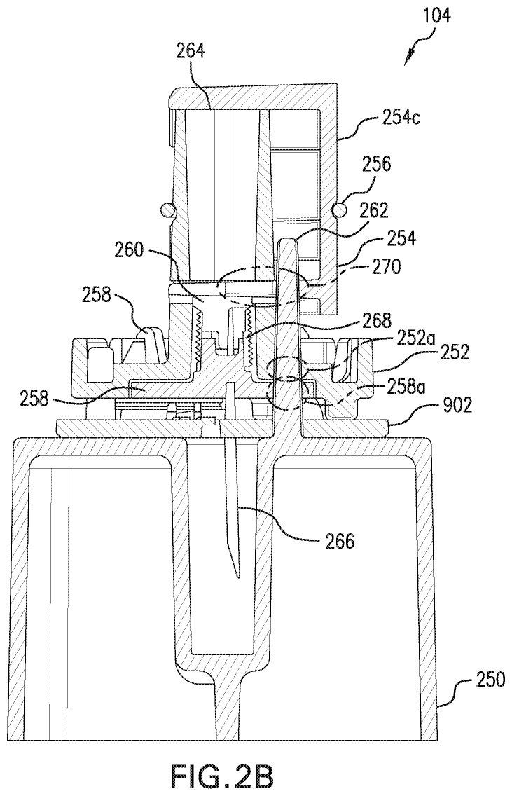

FIG. 2A is an exemplary cross-section view of select components of the reusable portion 102 in a first position, in accordance with embodiments of the present invention. The reusable portion 102 includes the following components: a housing 200, an actuator 202, a slider 204 and an insertion spring 206. The housing 200, shown as a cross-section to provide a better view of the other components with its interior, has a proximal end 208a and a distal end 208b. Located toward the proximal end 208 is a latch 212b. Located within an interior of the housing 200 is a retention wall 210. The actuator 202 includes an actuator face 216 that remains on an exterior of the housing 200. The actuator 202 further includes an actuator guide arm 218 and an actuator arm 220. Both the actuator arm 220 and actuator guide arm 218 connect to the actuator face 216 and portions of both the actuator arm 220 and the actuator guide arm 218 are located partially within an interior of the housing 200. In preferred embodiments, when the reusable portion 102 is in a second position, a user can displace or push the actuator face 216 resulting in displacement of the actuator arm 220 and actuator guide arm 218 within the housing.

The slider 204 fits within the interior of the housing 200. The slider 204 includes a tail 212a and a distal face 214. The slider 204 can be displaced within the housing 200 in a vertical direction (from the distal end 208b toward the proximal end 208a—and vice versa). The reusable portion 102 as illustrated in FIG. 2A is in a first position where the insertion spring 206 is shown as being minimally compressed. In this first position, the slider 204 is located toward the distal end 208b and prevented from being ejected from the housing 200 by the slide stop 222. Additionally, the insertion spring 206 may be exerting some force on the slider 204 such that the slider 204 is held firmly against the slide stop 222. Displacement of the slider 204 toward the proximal end 208a compresses the insertion spring 206. Sufficient compression of the insertion spring 206 results in the tail 212a being displaced over the latch 212b. When the tail 212a is captured on the latch 212b, the reusable portion 102 is considered in a second position. In the second position, the actuator arm 220 is in a position to horizontally displace the tail 212a thereby pushing/moving the tail 212a off the latch 212b. When the tail 212a is no longer retained by the latch 212b, the energy stored in the insertion spring 206 is released and is able to drive or displace the slider 204 toward the distal end 208b.

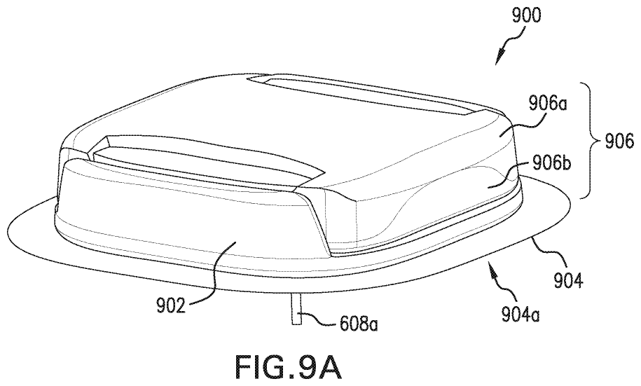

FIG. 2B is an exemplary cross-section view of the disposable portion 104 in the first state, in accordance with embodiments of the present invention. As illustrated, the disposable portion includes a base 902 that will be discussed more in depth in FIG. 9A. The entirety of the disposable proportion 104 is intended to be assembled and sterilized separately from the reusable portion 102.The disposable portion 104 includes a safety 250, a container 252, a translator 254, an o-ring 256, a distal end needle holder 258, a proximal end needle holder 260, a needle/sharp 266, and a retraction spring 268. The container 252 includes a container volume 264. The safety 250 includes a safety probe 262 that passes through a safety aperture 270 that is formed in the translator 254. Additionally, the safety 250 passes through a container safety aperture 252a, and a distal needle holder aperture 258a.

Select components of the disposable portion 104 are intended to be removably coupled with the reusable portion 102. Note, where discussion of the reusable portion 102 was made with regards to being in a first position and the second position, discussion of the disposable portion 104 will be regarding specific states rather than positions. When discussed together, states of the disposable portion 104 will be correlated with positions of the reusable portion 102. As illustrated in FIG. 2B, the disposable portion 104 is shown in the first state. In the first state the retraction spring 268 is compressed between the proximal end needle holder 260 and the distal end needle holder 258. Additionally, in the first state, the safety probe 262 extends through the container safety aperture 252a, the distal end needle holder aperture 258a, and the safety aperture 270. The safety probe 262 extending through the safety aperture 270 places the o-ring 256 in tension, or stretches the o-ring 256 against both the container 252 and the translator 254. Alternatively, the o-ring is applying a compression force against the container face 252a and the translator face 254c.

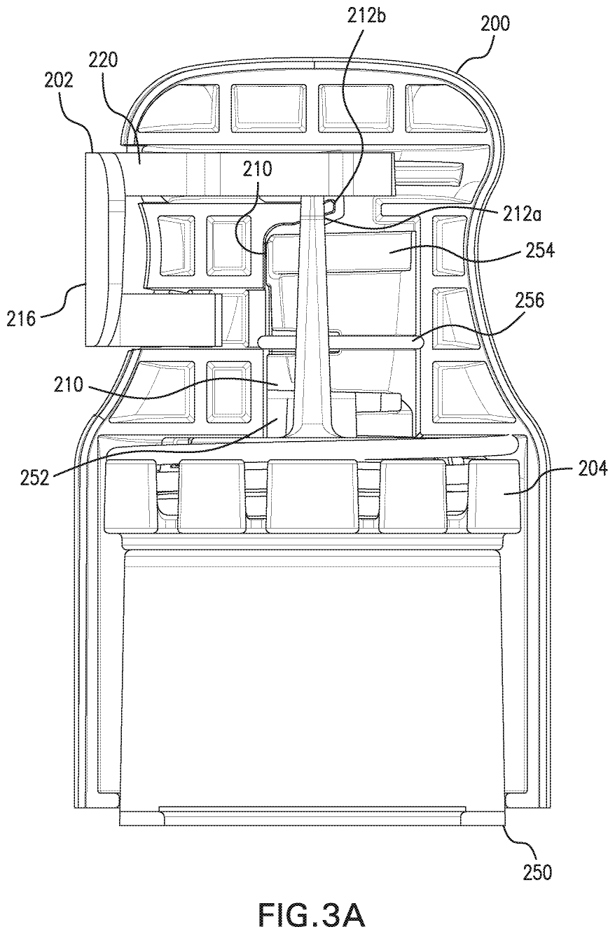

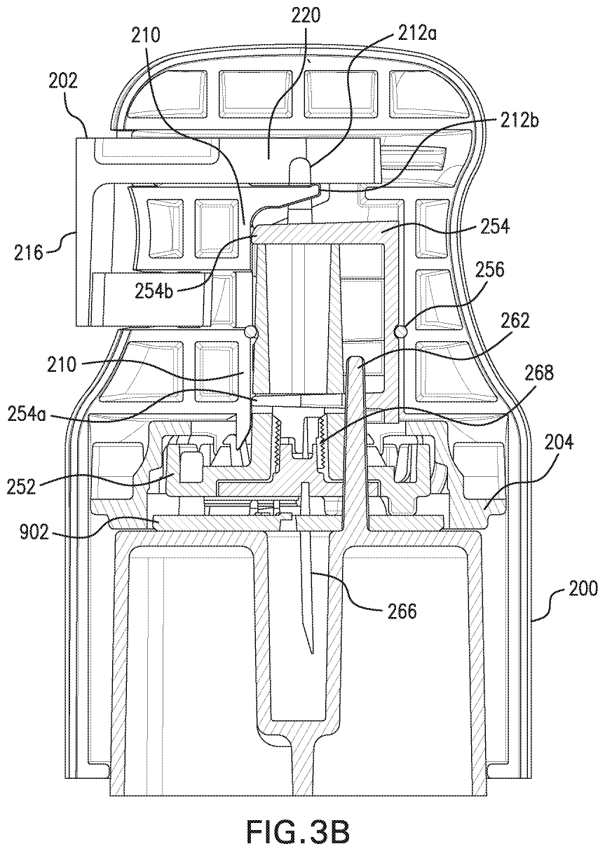

FIG. 3A is an exemplary illustration of a partial cross-section view of the reusable portion 102 in the second position while the disposable portion 104 is in the first state, in accordance with embodiments of the present invention. In preferred embodiments, getting the insertion device 100 into the position illustrated in FIG. 3A is accomplished by positioning the respective parts as illustrated in FIG. 1A and pressing the reusable portion 102 onto the disposable portion 104, thereby compressing the insertion spring (not shown), until the tail 212a is secured over the latch 212b. As illustrated, the tail 212a is shown being positioned over the latch 212b.

FIG. 3B is an exemplary illustration of a cross-section view of select parts of the reusable portion 102 in the second position and the disposable portion 104 in the first state, in accordance with embodiments of the present invention. For simplicity, the insertion spring 208 (FIG. 2A) is not shown but when the reusable portion 102 is in the second position. In the second position, the insertion spring 208 is in a compressed state. As illustrated, the actuator 202 is in a loaded position that enables portions of the actuator arm 220 to displace the tail 212a from its position over the latch 212b. In many embodiments, the actuator arm 202, or portions of the actuator arm 202, temporarily deform or plastically bends the tail 212a to displace the tail 212a from its location over the latch 212b. With the safety 250 as illustrated, the safety probe 262 is directly in contact with the container 252. Additionally, the safety probe 262 prevents the o-ring 256 from displacing the translator 254 via a compression force applied by the o-ring 256. In addition to the safety probe 262, also preventing the o-ring 256 from displacing the translator 254 is a retention wall 210. Specifically, translator faces 254a and 254b are prevented from lateral translation by their proximity to the retention wall 210. The needle/sharp 266 is illustrated being positioned through the base 902. Additionally, the retraction spring 268 is compressed.

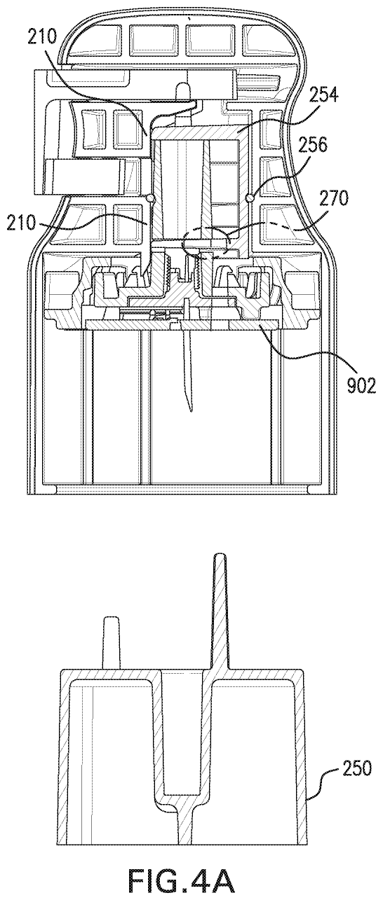

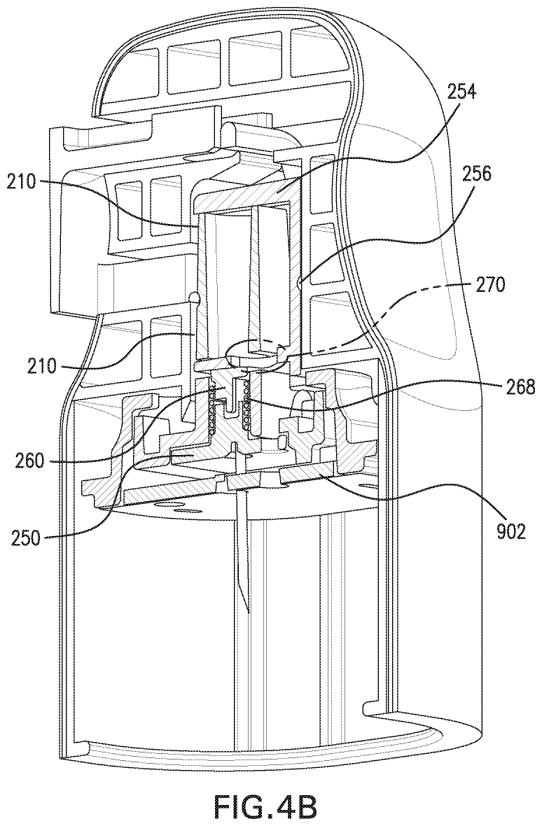

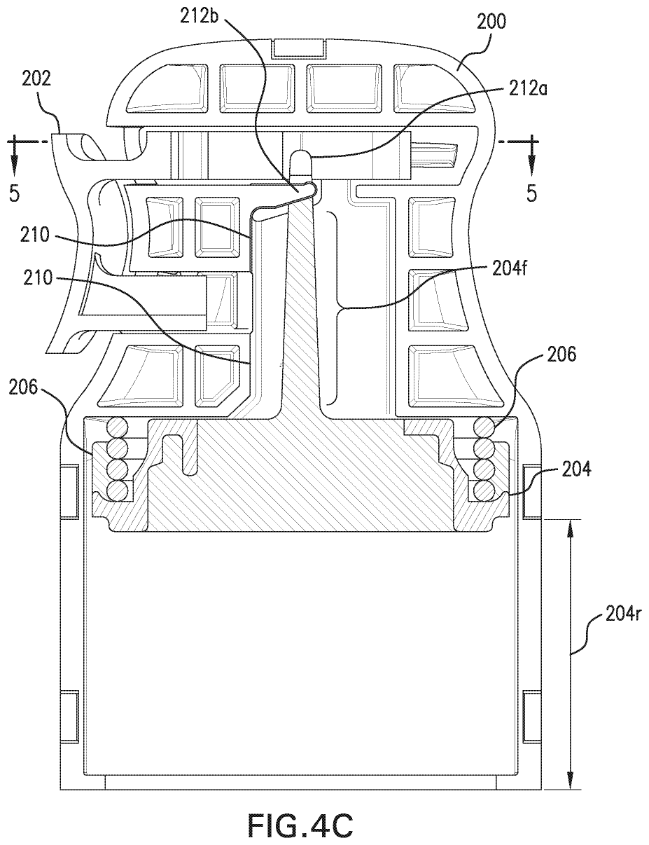

FIG. 4A is an exemplary cross-section view of the reusable portion 102 in the second position and the disposable portion 104 in a second state, in accordance with embodiments of the present invention. In the second state the safety 250 has been separated from the disposable portion 104. Removal of the safety 250 results in the retention wall 210 resisting compression force applied by the o-ring 256 onto the translator 254. Additionally, removal of the safety 250 also fully exposes an entirety of the safety aperture 270.

FIG. 4B is a pseudo-isometric cross-section view of the reusable portion 102 in the second position and the disposable portion 104 without the safety 250 in the second state, in accordance with embodiments of the present invention. As illustrated, the safety aperture 270 is an opening in the translator 254. The safety aperture 270 is sized to accommodate passage of the proximal end needle holder 260. However, as illustrated, in the second state the safety aperture 270 is misaligned with the proximal end needle holder 260. The misalignment between the safety aperture 270 and the proximal end needle holder 260 results in the retraction spring 268 being maintained in compression between the proximal end needle holder 260 and the distal end needle holder 258. Additionally, FIG. 4B further illustrates how the retention wall 210 interacts with the translator 254 to prevent the o-ring 256 from displacing the translator 254.

FIG. 4C is an exemplary illustration of a cross-section of select components of the reusable portion 102 in the second position, in accordance with embodiments of the present invention. The select components are the housing 200, the actuator 202 and the slider 204. In FIG. 4C the select components are illustrated in a second position, or loaded position- or in a position ready to perform an insertion of a device removably coupled to the slider 204 (not shown). Depressing the actuator 202 transitions the insertion device back to the first position. The insertion of the needle/sharp occurs during the transition from the second position to the first position. As the actuator is depressed, a face of the actuator pushes the tail 212a of the slider 204 beyond or over the latch 212b of the housing 200. In preferred embodiments, the tail 212a is a plastic part that flexes along a flex portion 204f of the slider 204.

Displacing the tail 212a beyond the latch 212b, releases energy stored in the insertion spring 206 located or positioned between the slider 204 and the housing 200. The release of the insertion spring 206 propels the slider 204 (and any attached components) through a slider range 204r within an interior of the housing 200.

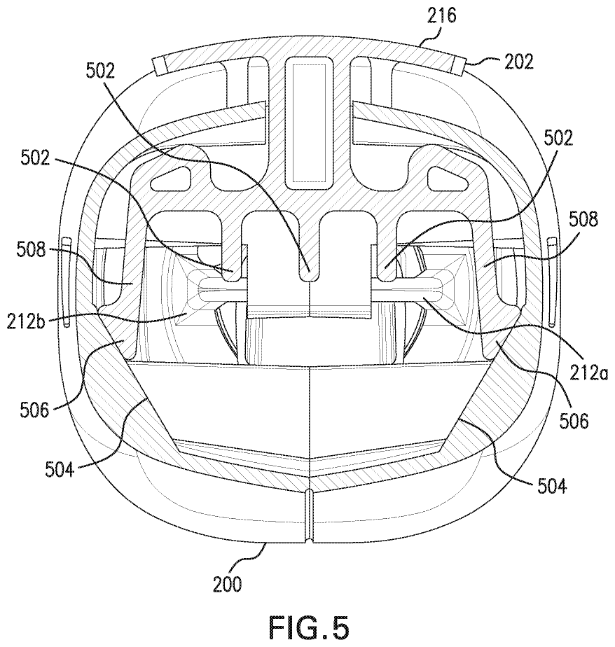

FIG. 5 is a cross-section view through the reusable portion 102 in the first position illustrating interactions between the actuator 202, the housing 200, the tail 212a and the latch 212b, in accordance with embodiments of the present invention. The view presented in FIG. 5 is a top view looking down with the cross-section view removing the proximal end 208a (FIG. 2A). As illustrated, the tail 212a is shown under the latch 212b, indicating the reusable portion 102 is in the first position. The view illustrated in FIG. 5 better illustrates features of both the actuator 202 and the housing 200. For example, multiple actuator pushers 502 are visible in FIG. 5. When the tail 212a is in the second position and located over (or upon) the latch 212b, the actuator pushers 502 are intended to interface with the tail 212a and upon application of force upon the actuator face 216, displace the actuator pushers 502 and subsequently the tail 212a off of the latch 212b. FIG. 5 further illustrates interactions between return arms 508 that include actuator return 506 and housing return 504. In many embodiments the housing return 504 is a face formed on the interior of the housing 200. The housing return interfaces with the actuator return 506 that is formed on the end of return arm 508. Depressing the actuator 216 applies a force that is transmitted through the return arm 508 to the actuator return 506 that is subsequently resisted by housing return 504. However, application of sufficient force to the actuator 216 when it is in the second position results in deflection of the return arms 508 via a force from the housing return 504 being applied to the actuator return 506, that deflects the actuator returns 506 toward the actuator pushers 502. After sufficient force has been applied to deflect the tail 212a off of the latch 212b (thereby releasing the slider from the second position so it can return to the first position), releasing the actuator 216 results in the housing return 504 applying a force through the actuator return 506 that returns the actuator 202 to the first position.

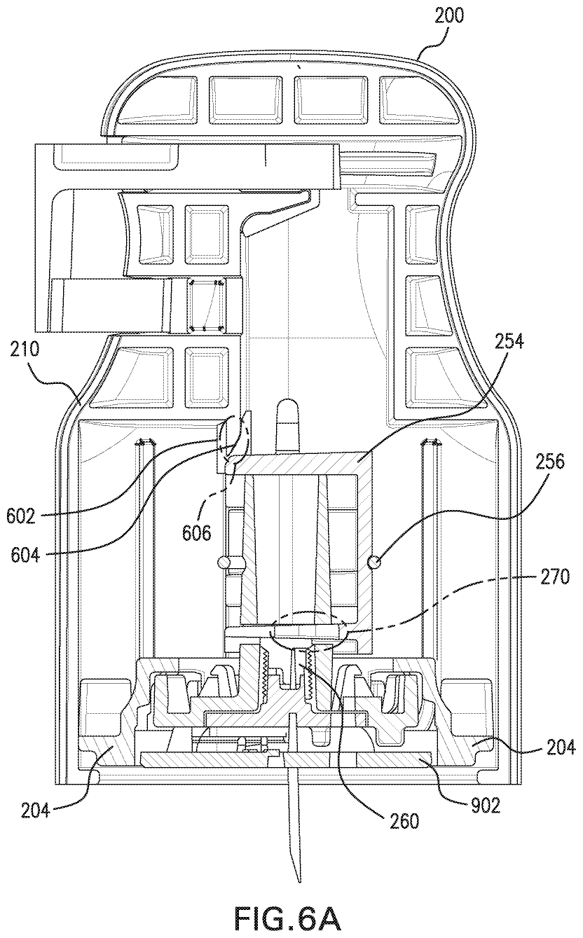

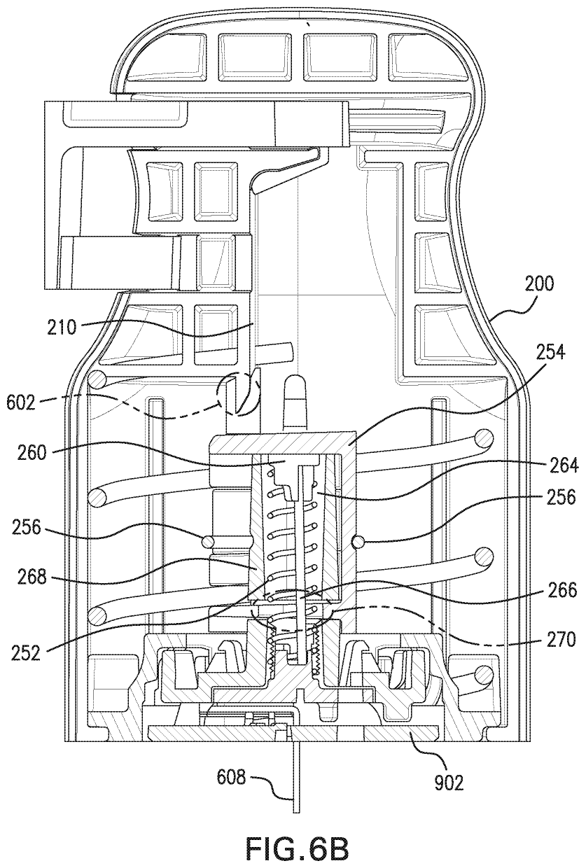

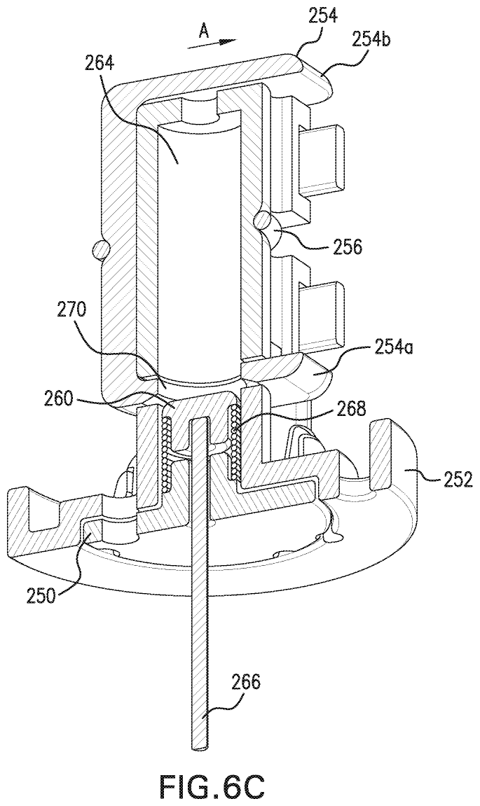

FIG. 6A is an exemplary cross-section view of select parts of the reusable portion 102 (without the insertion spring) in an intermediary position between the first position and the second position, and select parts of the disposable portion 104 in a third state, in accordance with embodiments of the present invention. In FIG. 6A the slider 204 is in an intermediate position between the first position and the second position. The intermediate position illustrated is intended to show the interaction between the translator 254 and the retention wall 210 along with retraction timing 602. Note that retraction timing 602 extends away or protrudes from the retention wall 210 toward the distal end of the housing 200. Additionally, the retraction timing 602 further incorporates retraction lead-in 604. Both the retraction timing 602 and retraction lead-in 604 interact with the translator 254 to accomplish delaying when horizontal movement of the translator 254 is allowed. In the second position and second state, the translator 254 is restricted to vertical displacement or movement because the compression force exerted by the o-ring 256 is resisted by the retention wall 210 being positioned to the left side of the translator 254. As illustrated, the translator 254 has traversed the vertical length of the retention wall 210 along with the retraction timing 602. Accordingly, compression applied by the o-ring 256 is capable of imparting horizontal displacement/translation of the translator 254 toward the left. In many embodiments, the translator 254 includes a translator lead-in 604 that compliments the retention lead-in 604 to enable a smoother initiation of horizontal movement of the translator 254. In FIG. 6A, the safety aperture 270 remains unaligned with the proximal end needle holder 260.

FIG. 6B is an exemplary cross-section view of select portions of the reusable portion 102 in the first position and select portions of the disposable portion 104 in a fourth state, in accordance with embodiments of the present invention. In FIG. 6B, select parts of the reusable portion 102 have returned to the first position. Additionally, the translator 254, having traversed the retention wall 210 and retention timing 602, has been horizontally displaced by the compression of the o-ring 256. Moreover, the horizontal displacement of the translator 254 axially aligned the safety aperture 270 with the proximal end needle holder 260. Upon axial alignment of the safety aperture 270 with the proximal end needle holder 260, the retraction spring 268 is capable of expanding or decompressing. Expansion of the retraction spring 268 results in vertical displacement of the proximal end needle holder 260 upward (toward the proximal end of the housing 200), and into the container volume 264 of the container 252. As the needle/sharp 266 is retained or coupled to the proximal end needle holder 260, the needle/sharp 266 is also displaced into the container volume 264. FIG. 6B further illustrates the base 902 being located at the distal end of the housing 200. Additionally, a sensor 608 that was previously within the needle/sharp 266 is exposed.

FIG. 6C is an exemplary pseudo-isometric cross-section illustration of select parts of the disposable components 104 of the insertion device in an intermediate position, between the third state (FIG. 6A) and the fourth state (FIG. 6B), in accordance with embodiments of the present invention. Specifically, the housing, the slider and actuator have been removed from FIG. 6C. The components illustrated in FIG. 6C also represent the majority of the components that are sterilized; the missing sterilized components include the safety, the sensor, and the sensor base. FIG. 6C helps illustrate interactions between the distal end needle holder 250, the proximal end needle holder 260, the container 252, the translator 254, and the o-ring 256.

Specifically, FIG. 6C is an illustration of when compression applied by the o-ring 256 has laterally shifted or moved the translator 254 in direction A, resulting in the safety aperture 270 being axially aligned with the proximal end needle holder 260. In FIG. 6C, the transitional state between the third state and the fourth state exemplary illustrates the moment before the retraction spring 268, shown being compressed between the proximal needle holder 260 and the distal end needle holder 258, is unloaded and results in the proximal end needle holder 260 and the attached or coupled needle or sharp 266 being retracted into the container volume 264.

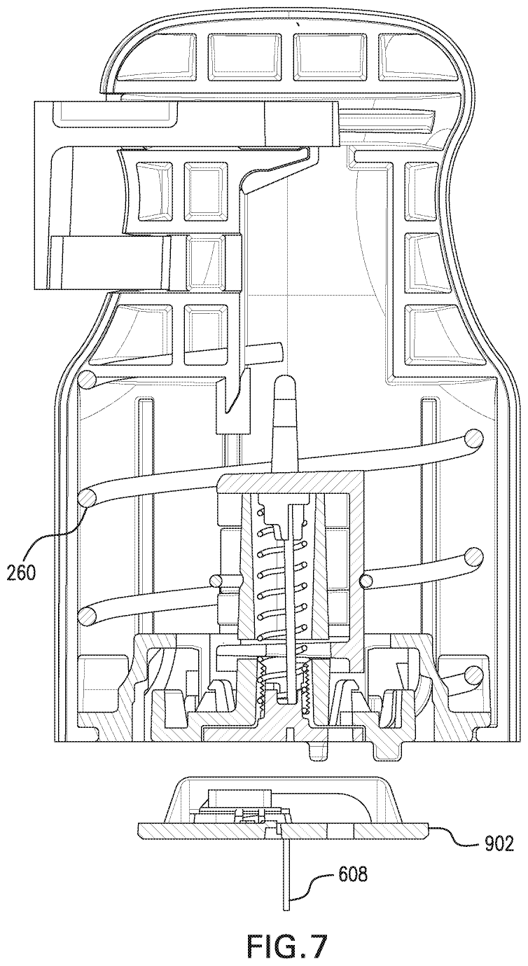

FIG. 7 is an exemplary cross-section view of portions of the reusable portion 102 in the first position and portions of the disposable portion 104 in a fifth state, in accordance with embodiments of the present invention. In the fifth state, the base 902 has been removably coupled to a subject and the reusable portion 102 and remainder of the disposable portion 104 are lifted away from the base 902.

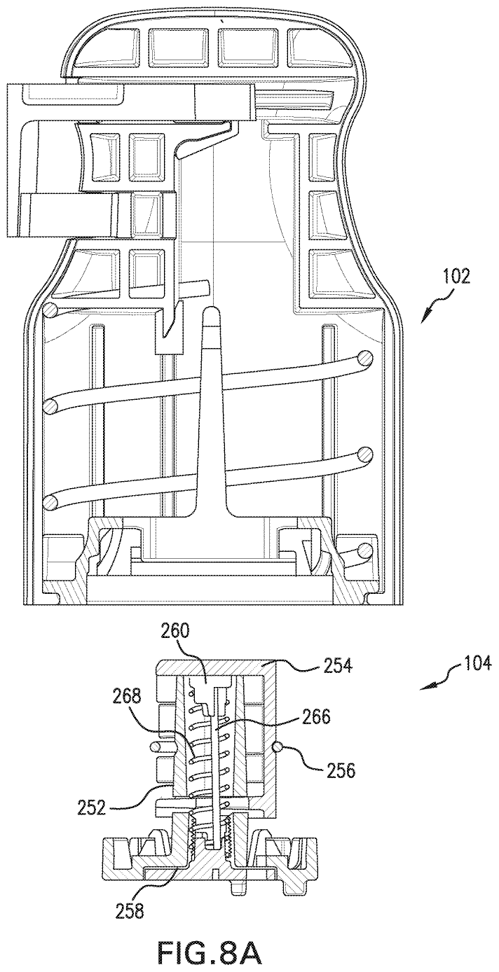

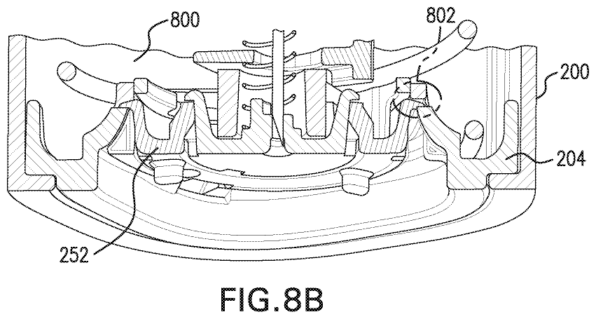

FIG. 8A is an exemplary cross-section view of select components of the disposable 104 portion being detached or separated from select portions of the reusable portion 102, in accordance with embodiments of the present invention. The select components of the disposable portion 104 include the translator 254, the container 252, the proximal end needle holder 260, the needle/sharp 266, the retraction spring 268, the o-ring 256 and the distal end needle holder 258.

FIG. 8B is an exemplary cross-section view of select components of the disposable portion 104 interfaced with select components of the reusable portion 102, in accordance with embodiments of the present invention. In preferred embodiments, the container 252 includes at least one container latch 800. The container latch 800 is intended to interface with a slider retainer 802 that is formed on the slider 204. In many embodiments, the container latch 800 is coupled to the slider retainer 802 when the reusable portion 102 is pushed onto the disposable portion 104, thereby “loading” the insertion device 100. In many embodiments, the container latch 800 and accordingly the container 252 itself can be decoupled from the slider retainer 802 by applying a twist of the container 252 relative to the slider 204.

FIG. 9A is an exemplary pseudo-isometric view of an on-body assembly 900, in accordance with embodiments of the present invention. As illustrated, the on-body assembly includes a patch 904 that is coupled to the base 902. The patch 904 has a skin-side 904a and a base side 904b. Adhesive on the skin-side 904a can temporarily secure the patch 904 and base 902 to a user. Adhesive couples the base 902 to the base side 904b of the patch 904.The base 902 is configured to removably couple with an electronics module 906. The electronics module 906 that is removably coupled to the base 902 includes both a top case 906a and a bottom case 906b. FIG. 9A also includes an illustration of a distal end 608a of the sensor 608.A proximal end 608b (not shown) of the sensor 608 is located or positioned on the base 902. Coupling the electronics module 906 to the base 902 enables electrical contact between a power source within the electronics module 906 and the proximal end 608b positioned on the base 902.

FIG. 9B is a top view of the on-body assembly 900, in accordance with embodiments of the present invention. The top case 906a of the electronics module 906 is visible. Additionally, small portions of the base 902 are visible extending beyond the top case 906a. Furthermore, the patch 904 is visible surrounding the base 902 and electronics module 906. The extension of the patch 904 beyond a perimeter of the base 902 is exemplary and provided for sake of illustration. In other embodiments, the patch 904 does not extend past or beyond the perimeter of the base 902.

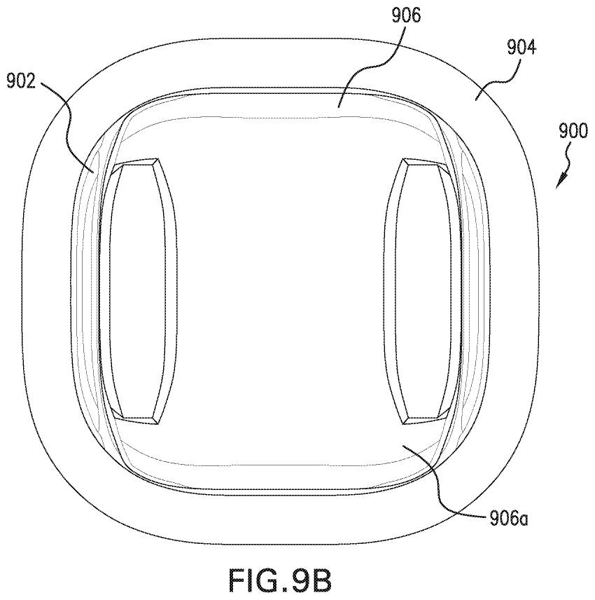

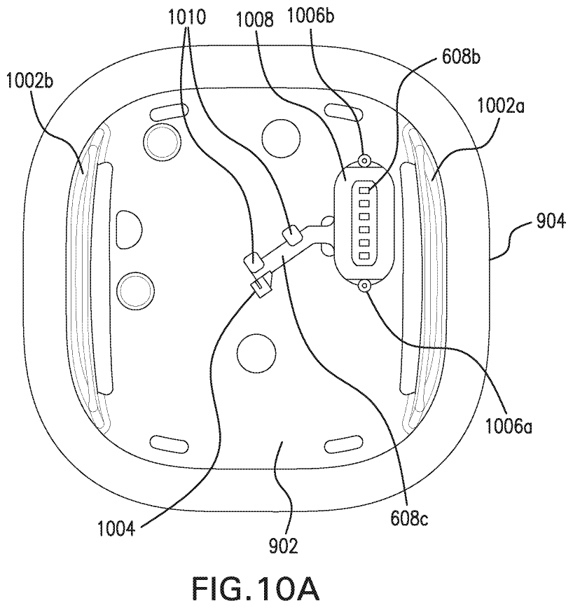

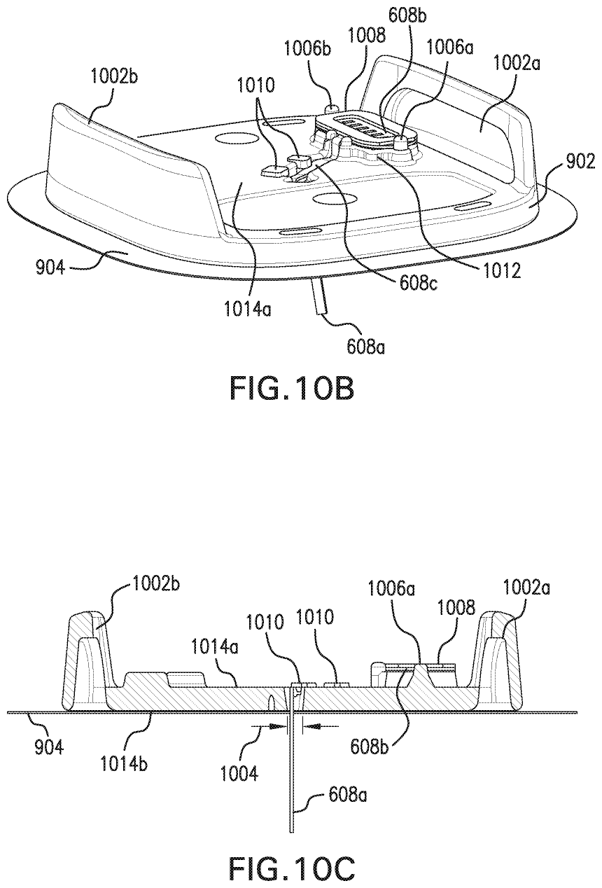

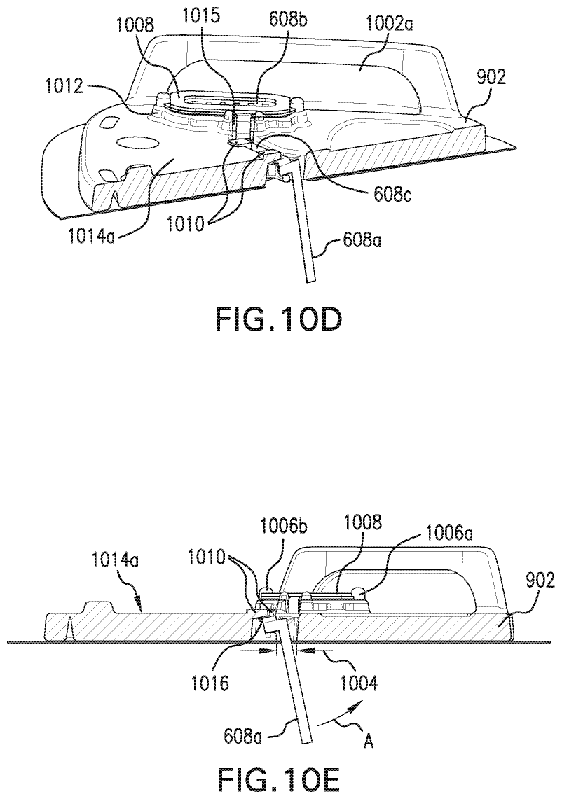

FIG. 10A is a top view of the base 902, in accordance with embodiments of the present invention. The base 902 includes clips 1002a and 1002b that enable the electronics module (not shown) to be removably coupled to the base 902. The sensor 608 has a midportion 608c and proximal end 608b being coupled, retained or mounted onto the base 902. Retention tabs 1010 secure the midportion 608c of the sensor 608 to the base 902. The base 902 further includes proximal locating features 1006a and 100b that assist in locating or positioning the proximal end 608b to a specific position or location on the base 902. Located or positioned over the proximal end 608b is a base gasket 1008. The base gasket 1008 may be coupled to the proximal end 608b using an adhesive. Additionally, the proximal end 608b can be coupled to the base 902. In preferred embodiments, adhesives are used to couple the proximal end 608b to the base 902 and the base gasket 1008 to the proximal end 608b. Alternatively, the base basket 1008 may be held in place over the proximal end 608b via compression from the electronics module (not shown). The base 902 also includes an aperture 1004 that allows both the sensor 608 and the needle/sharp (not shown, from the insertion assembly) to pass through the base 902 and patch 904.

FIG. 10B is a pseudo-isometric view of the base 902, in accordance with embodiments of the present invention. The base 902 includes an interior 1014a. The retention tabs 1010 protrude or extend away from the interior 1014a. Additionally, a sensor platform 1012 and the proximal locating features 1006a and 1006b extend or protrude away or above from the interior 1014a. FIG. 10B also provides an illustration of the clip 1002a. As illustrated, the clip 1002a is an undercut within a vertical wall that extends vertically away from the interior base 1014a. The clip 1002a are shaped to accommodate features of the electronics module 906.

While the embodiment illustrated in FIG. 10B includes retention tabs 1010, other embodiments may have fewer or more retention tabs 1010. The retention tabs 1010 help secure or retain the midportion 608c of the sensor to the base 902. The retention 1010 work in conjunction with additional features within the base 902 to position the distal end 608a of the sensor.

FIG. 10C is an exemplary cross-section of the base 902 that illustrates the aperture 1004 traversing through the base 902 from the interior 1014a to an exterior 1014b, in accordance with embodiments of the present invention. The aperture 1004 allows both the sensor 608 and needle/sharp (not shown) from the insertion device to be extended through the base 902. FIG. 10C further illustrates the clips 1002a and 1002b that are formed as part of the base 902. The clips 1002a and 1002b are undercuts formed in a wall that extends vertically away from the interior base 1014a. Both clips 1002a and 1002b accommodate features from the electronics module 906. The interaction between the features from the electronics module 906 enable removable coupling between the electronics 906 and the base 902.

FIG. 10D is an exemplary pseudo-isometric cross-section of the base 902 that illustrates how the portions of the sensor 608 interact with the base 902 to influence positioning of the distal end 608a as it protrudes through the base aperture 1004, in accordance with embodiments of the present invention. In preferred embodiments the sensor 608 is flexible and allows some bending and twisting of the sensor 608 without compromising operational functionality. For example, the proximal end 608b of the sensor is coupled to the sensor platform 1012 and the mid portion 608c of the sensor is secured within a sensor channel 1015 formed in the interior base 1014a using the retention tabs 1010. Accordingly, the sensor 608 must be able to bend or flex from the sensor platform 1012 toward the sensor channel 1015 formed in the interior base 1014a. Additionally, FIG. 10D provides an exemplary illustration of features of the base 902 applying a bias to the sensor that results in the distal end 608a being canted or biased in a preferred direction as it protrudes through the base 902. In preferred embodiments, the features of the base 902 that impart are bias to the sensor 608 include the sensor channel 1015, the retention tabs 1010 and a bias face 1016 that will be discussed in more detail below.

FIG. 10E is an exemplary cross-section of the base 902 that illustrates a bias face 1016 formed in the sensor channel 1015 that biases the sensor 608 at an angle away from vertical (as illustrated), in accordance with embodiments of the present invention. The bias face 1016 formed at the bottom of the sensor channel 1015 is intended to apply a slight twist to the mid portion 608c of the sensor (axis of rotation being normal to the page) when the midportion 608c of the sensor 608 within the sensor channel 1015 is placed under the retention tabs 1010. As illustrated, the mid portion 608c of the sensor 608 being twisted results in the distal end 608a of the sensor being biased away from perpendicular to the proximal end 608b of the sensor. For example, when the mid portion 608c is held against the bias face 1016 by the retention tabs 1010, the slight twist or bias on the mid portion 608c results in the distal end 608a of the sensor 608 rotating counterclockwise in direction A. An advantage of applying the bias to the distal end 608a of the sensor is to ensure the distal end 608a is retained within the interior of the sharp 266 (FIG. 2B) of the disposable portion 104. In preferred embodiments, the sharp 266 includes a channel that extends the length of the sharp. Without biasing the distal end 608a against the sharp, it is more likely that the distal end 608a can be ejected from the sharp resulting in the sensor not being properly inserted. Biasing the distal end 608a toward the sharp improves the likelihood the distal end 608a is inserted to the preferred depth and the sensor does not suffer damage during the insertion procedure.

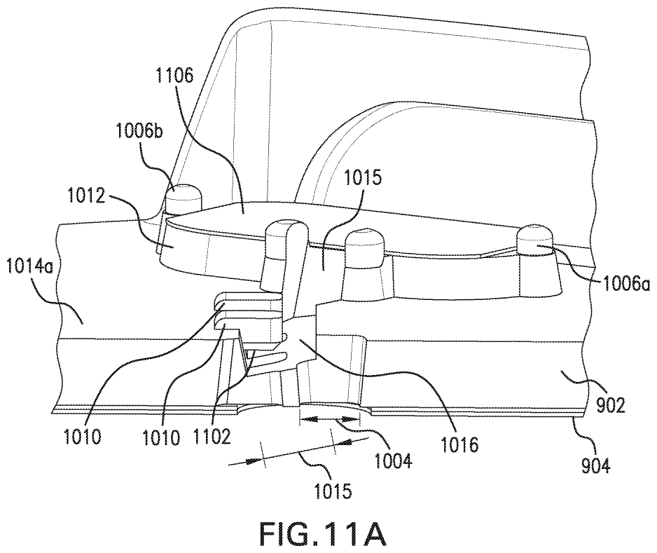

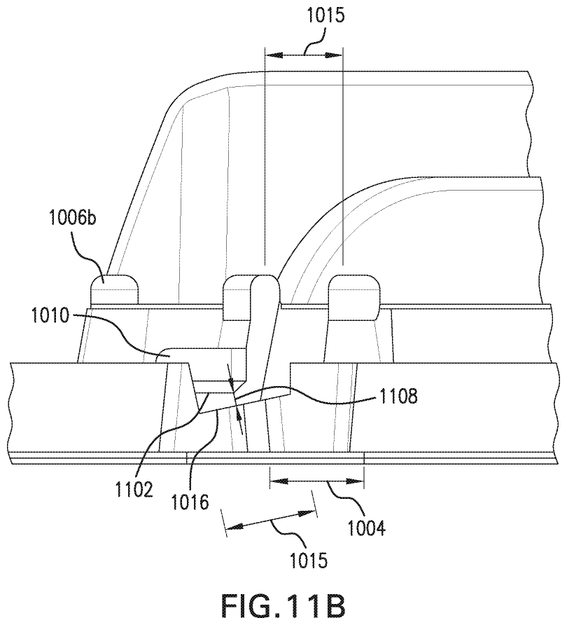

FIG. 11A is a pseudo-isometric cross-section view of the base 902, the patch 904 and an adhesive 1106, in accordance with embodiments of the present invention. The adhesive 1106 is located on the sensor platform 1012 and couples the sensor (not shown) to the sensor platform 1012. Without the sensor, the sensor channel 1015 is more visible. The sensor channel 1015 is a channel that extends away from the sensor platform 1012 while also being positioned below the interior base 1014a. The retention tabs 1010 include a portion formed above the interior base 1014a. Additionally, the retention tabs 1010 include a retention face 1102 formed above the bias face 1016 within the sensor channel 1015. Though illustrated as being substantially parallel with the interior base 1014a, the retention face 1102 may be formed at different angles relative to either the interior base 1014a or the sensor channel 1015. For example, in some embodiments the retention face 1102 may be substantially parallel or convergent with the bias face 1016.

FIG. 11B is a cross-section view of the base 902 illustrating the interaction between the bias face 1016, the sensor channel 1015 and the retention face 1102 of the retention tab 1010, in accordance with embodiments of the present invention. A retention gap 1108 is visible in FIG. 11B and is defined as a distance between the retention face 1102 and the bias face 1016. In various embodiments, the retention gap 1108 may be modified by adjusting a variety of dimensions including, but not limited to how far the retention tab 1010 extends over the sensor channel 1015, an angle of the bias face 1016 relative to the retention tab 1010 and the like.

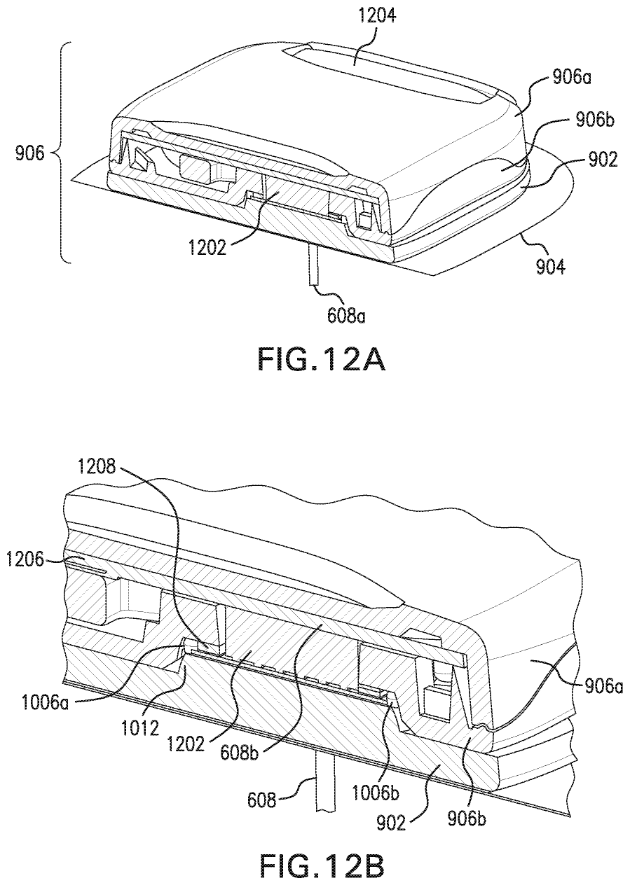

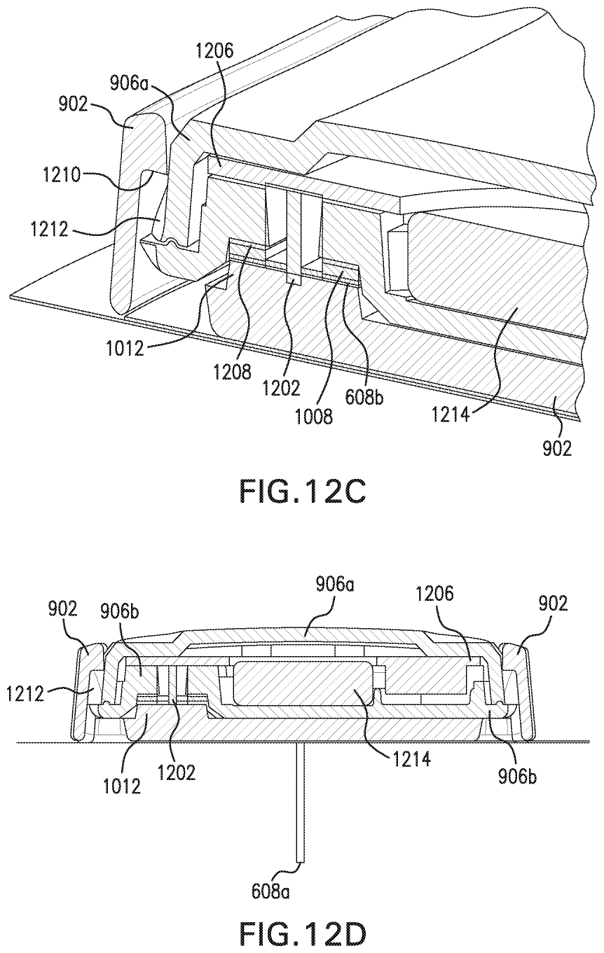

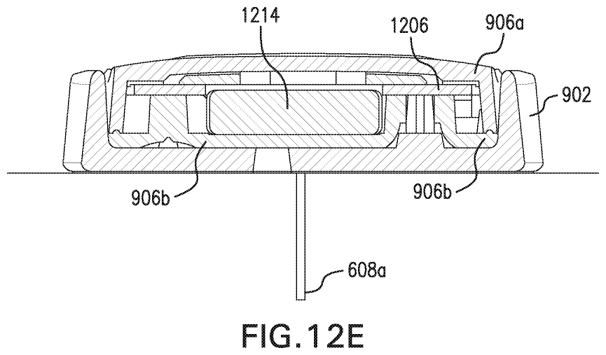

FIGS. 12A-12C are exemplary pseudo-isometric cross-sections of the electronics module 906 that also includes the base 902, patch 904, in accordance with embodiments of the present invention. The electronics module 906 includes the bottom case 906b and top case 906a. The cross-section illustration includes portions of the base 902 like the sensor platform 1012, the proximal location features 1006a and 1006b, the proximal end 608b of the sensor 608, and the base gasket 1008. Retained between the top case 906a and bottom case 906b of the electronics module 906 is a circuit board 1296. Making electrical connections between electrical contacts on the circuit board 1206 and the proximal end 608b of the sensor 608 is a conductor 1202 (black rectangle). In many embodiments the conductor 1202 is an elastomeric z-axis conductor. Also visible in FIGS. 12C and 12D are how a clip 1210, formed as part of the base 902 interacts with a flange 1212. The flange 1212 is a feature that is formed as part of the top case 906a. The flange 1212 interacts with the clip 1210 to removably couple the electronics module 906 to the base 902. FIG. 7C further illustrates an interaction between the base gasket 1008 and electronics module gasket 1208. In preferred embodiments the base gasket 1008 and the electronics module gasket 1208 are compliant materials such as, but not limited to rubber or elastomeric foams. The base gasket 1008 is coupled to the base 902 while the electronic module gasket 1208 is coupled to the bottom case 906b of the electronics module 906. The base gasket 1008 is compressed against the electronic module gasket 1208 when the electronics module 906 is coupled to the base 902. Compression of both the base gasket 1008 and the electronics module gasket 1208 forms a barrier to prevent any liquid that wicks up along the sensor 608 from entering an interior of the electronics module 906. The barrier formed between the base gasket 1008 and the electronic module gasket 1208 isolates the proximal end 608b of the sensor 608 on the sensor platform 1012 while sealing the sensor 608 within the sensor channel 1015 (FIG. 11B). Additionally, the barrier is formed between the electronics module 906 and the base 902. In many embodiments the distal end 608a of the sensor 608 is implanted into subcutaneous tissue. In some instances, during or after insertion of the sensor 608, bodily fluids like blood can be wicked up along the sensor 608. The barrier formed from the combined base gasket 1008 and electronics module gasket 1208 can prevent any bodily fluid that is wicked through or along the sensor channel 1015 (not shown) from reaching the electrical connection of the sensor 608 or circuit board 1206.

FIGS. 12D and 12E are cross-section views of the base, electronics module and patch from different perspectives in accordance with embodiments of the present invention. FIG. 12D illustrates interactions between the base 902, the top case 906a and the bottom case 906b. The flange 1212 can be seen on an outer wall of the top case 906a interacting with the base 902. Additionally, the circuit board 1206 can be seen captured between the bottom case 906b and the top case 906a. In some embodiments, the capturing of the circuit board 1206 between the top case 906a and the bottom case 906b imparts a compression force on the conductor 1202. In embodiments where the conductor 1202 is a conductive elastomer, the compression force makes an electrically conductive connection between the circuit board 1206 and the proximal end 608b of the sensor 608. A power supply 1214 is also visible in FIGS. 12D and 12E. In many embodiments the power supply 1214 is a battery that is mounted to the circuit board 1206.

In some embodiments, a flexible material such as foam or an elastomer is applied between the top case 906a and the circuit board 1206 to ensure a sufficient compression force between the circuit board 1206, the conductor 1202 and the proximal end 608b. In other embodiments, the stack up of components within the electronics module 906 is sufficient to make a secure and reliable connection between the proximal end 608b, the conductor 1202 and the circuit board 1206.

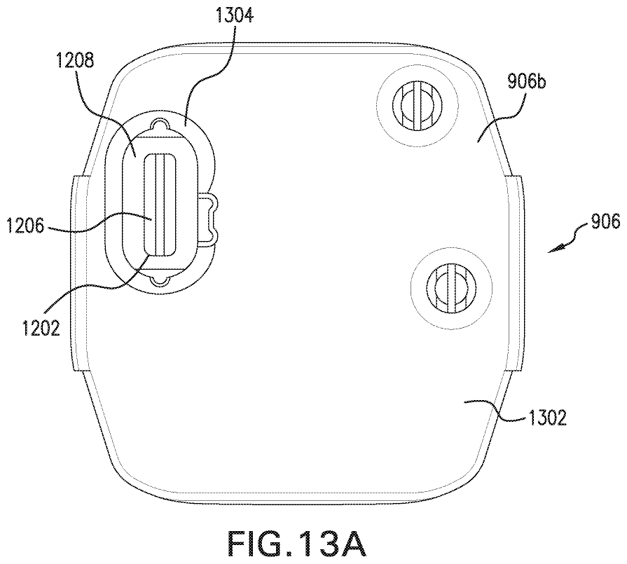

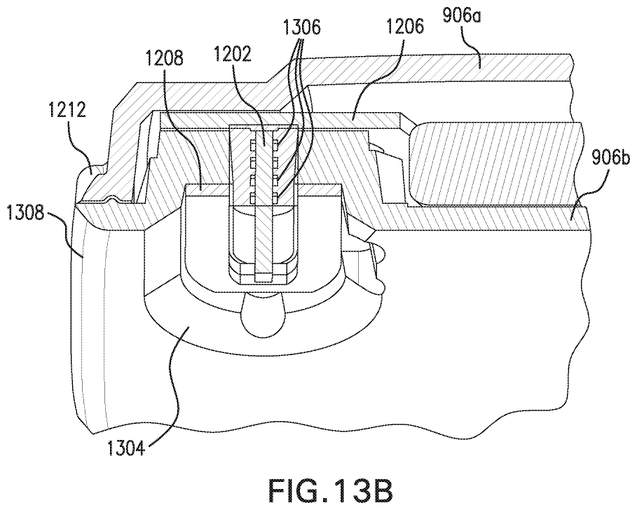

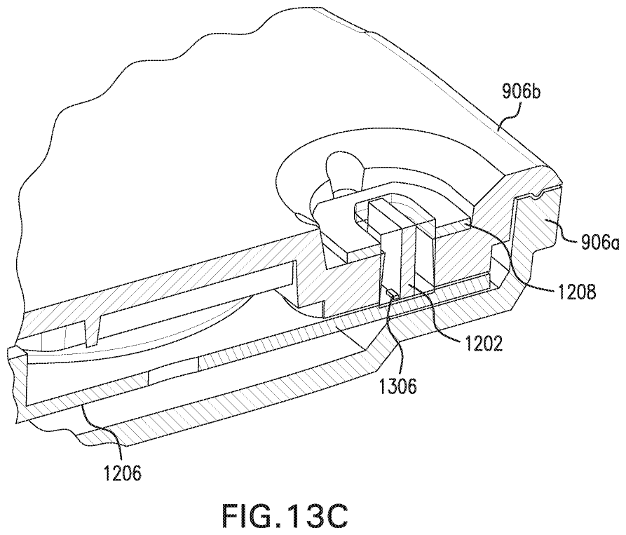

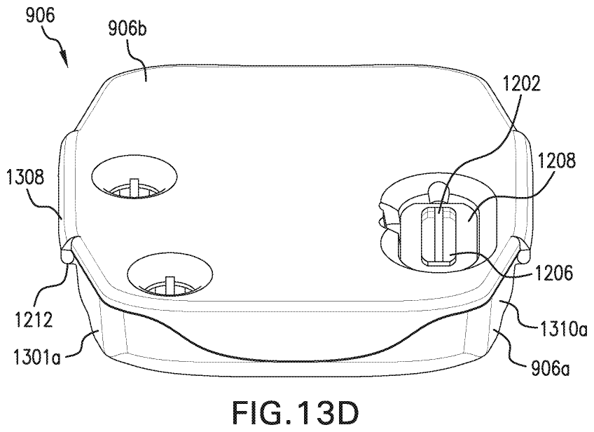

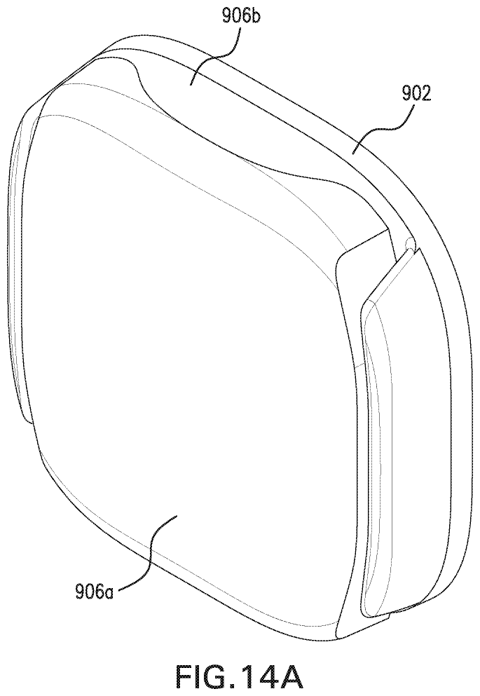

FIG. 13A is a view of a bottom face 1302 of the bottom case 906b of the electronics module 906, in accordance with embodiments of the present invention. The bottom face 1302 includes an interface aperture 1304 that exposes an interior portion of the electronics module 906. When the electronics module 906 is coupled to the base 902 (not shown), the interface aperture 1304 accommodates the sensor platform 1012 (not shown) of the base 902 (not shown). The conductor 1202 located within the interface aperture 1304 enables electrical connection between the electronics module 906 and the proximal end 608b of the sensor 608 that is mounted to the base 902. Within the interface aperture 1304, the electronic module gasket 1208 is visible along with the circuit board 1206 and the conductor 1202.

FIGS. 13B and 13C are pseudo-isometric cross-section views of the electronics module 906, in accordance with embodiments of the present invention. The cross-section illustrates the location of various components within the interface aperture 1304 that is formed between the bottom face 1302 of the bottom case 906b and the interior of the electronics module 906. For example, FIG. 13B includes the electronic module gasket 1208, the conductor 1202, the circuit board 1206 and electrical contacts 1306 on the circuit board 1206. In many embodiments, the conductor 1202 is a flexible z-axis conductor. The cross-section illustrated in FIG. 13B also illustrates a lead-in edge 1308 formed on the bottom case 906b and the flange 1212 formed on the top case 906a. The lead-in edge 1308 fits within the clip 1208 (not shown) of the base 902 (not shown). Additionally, in many embodiments the flange 1212 may be flush within the clip 1308 to form an edge of the electronics module 906. FIGS. 12C and 12D provide additional illustration of the clip 1210 and its interaction with both the flange 1212 and the bottom case 906b. In FIGS. 12C and 12D, the clip 1210 is illustrated as an undercut formed on a vertical wall that protrudes or extends away from the interior base 1014a.

FIG. 13D is a pseudo-isometric view of the electronics module 906 in accordance with embodiments of the present invention. Both of a first face 1310a and a second face 1310b of the electronics module 906, include the flange 1212 that is formed on the top case 906a and lead-in 1308 formed on the bottom case 906b.

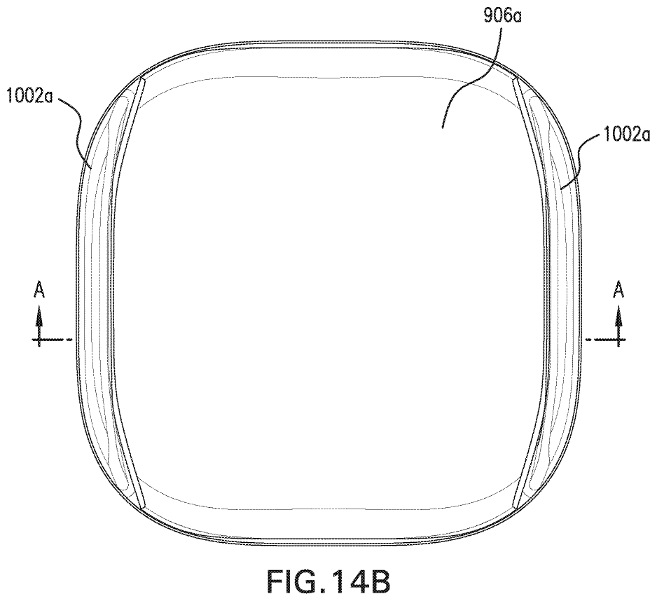

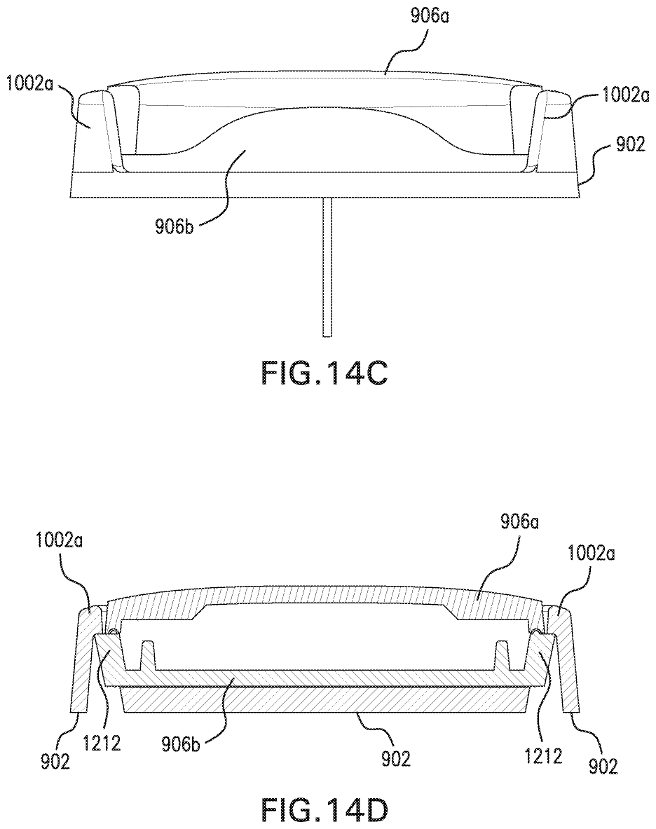

FIGS. 14A-14D are various illustrations of an alternative embodiment of select elements of the on body assembly 900 that includes the electronics module 906 mounted to the base 902 in accordance with embodiments of the present invention. FIG. 14A is a pseudo-isometric view the both the electronics module 906 and the base 902 (without the patch). Visible in FIG. 14A are the top case 906a, the bottom case 906b and the base 902. Also visible in FIG. 14A is the wall of the base 902 that includes the clip 1002a or 1002b. FIG. 14C is an exemplary view of the on body assembly 900 that illustrates the relative positions of the base 902, the top case 906a, the bottom case 906b and the sensor 608.

FIG. 14B is a top view of select components or elements of the on body assembly 900 showing section line A-A that was used to illustrate the cross-section view shown in FIG. 14D. FIG. 4D is cross-section of the on body assembly 900 illustrating how the alternative embodiment enables the clips 1002a and 1002b of the base 902 to interact with the bottom case 906b. In FIG. 14D, the base 902 is removably coupled to the electronics module 906 by a flange 1402 that is formed on the bottom case 906b. The embodiment illustrated in FIGS. 14A-14D is illustrates that clips formed on the base 902 can secure the electronics module 906 using one or more flanges formed on the bottom case 906b. This embodiment is different than previously illustrated embodiments where the flange is formed on the top case 906a.

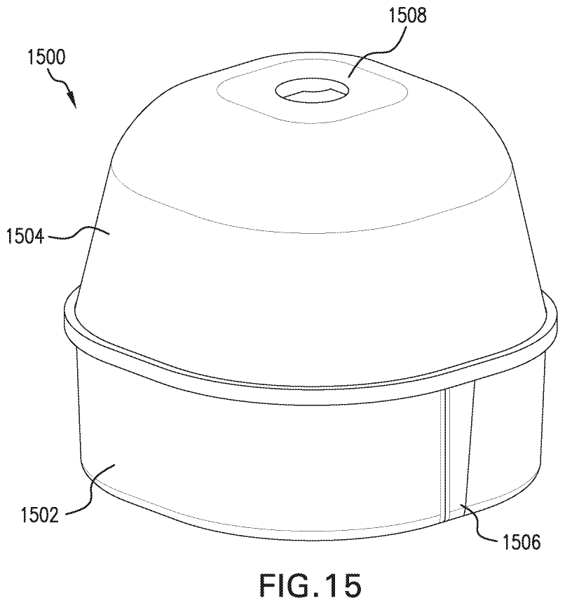

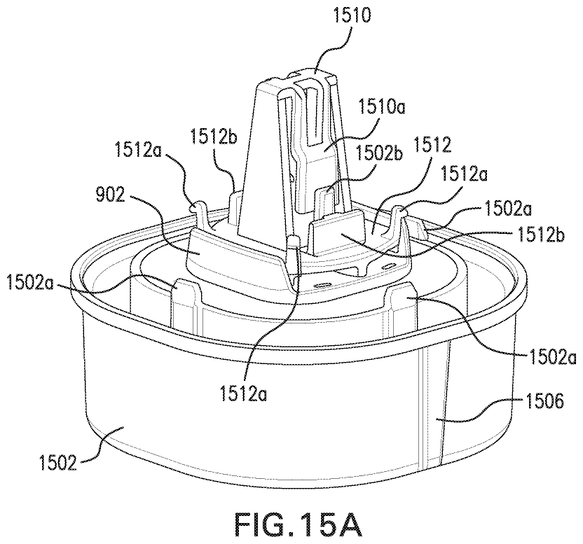

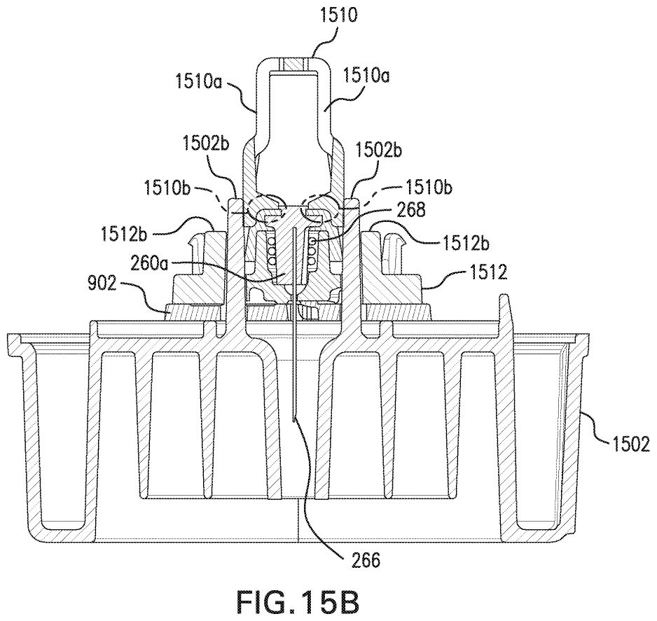

FIG. 15 is an isometric view of a sensor assembly 1500, in accordance with embodiments of the present invention. The sensor assembly 1500 includes a stand 1502 and a frame 1504 that is removably coupled to the stand 1502. The Stand 1502 further includes an alignment feature 1506 that is formed on an exterior wall of the stand 1502. The frame 1504 includes an aperture 1508 formed on a top surface of the frame 1504. In many embodiments the aperture 1508 passes through the frame 1504 thereby providing access from the exterior of the frame 1504 to an interior of the sensor assembly 1500.

FIG. 15A is an isometric view of select components of the sensor assembly 1500, in accordance with embodiments of the present invention. In FIG. 15A the frame 1504 has been removed revealing additional components included in the sensor assembly 1500 in a first state. In addition to alignment feature 1506, the stand 1502 further includes at least one or more key features 1502a. The key features 1502a can help ensure a proper orientation between the sensor assembly 1500 and the insertion assembly 1600 with each key feature 1502a being keyed to a corresponding feature on the insertion assembly 1600. On the stand 1502 is the base 902. In the first state, the base 902 is coupled to the patch 904 and detachably coupled to the top 1512. The retractor 1510 is detachably coupled to the top 1512. The stand 1502 further includes a post 1502b that is located between the retractor 1510 and the top 1512.

The top 1512 includes at least one top clip 1512a. In the embodiment illustrated in FIG. 15A, the top 1512 includes four top clips 1512a though only three top clips 1512a are visible. The top clips 1512a enable removable coupling of the base 902 and subsequently coupled elements or components to the insertion assembly 1600. Also formed on the top 1512 is at least a support 1512b. As shown in FIG. 15A, two supports 1512b are visible. Detachably coupled to the top 1512 is the retractor 1510. The retractor 1510 includes retractor arm 1510a.

FIG. 15B is a cross-section view of select components of the sensor assembly 1500 in a first state, in accordance with embodiments of the present invention. FIG. 15B illustrates interactions between the stand 1502, the base 902, the top 1512, the retractor 1510 along with the needle 266, the needle holder 260 and the retraction spring 268. The cross-section illustrates that in the first position, the stand 1502 extends below the needle 266 to prevent the needle 266 from unintentionally puncturing a person or object. Additionally visible is the location of the posts 1502b relative to the other components of the sensor assembly 1500. The posts 1502b extend through the patch, the base 902, the top 1512. The posts 1502b are located between both the supports 1512b and the retractor arms 1510a. As illustrated in the first state, the posts 1502b maintain or constrict the retractor arms 1510a in a compressed state.

The cross-section further illustrates that the retractor arms 1510a have at least one retractor clip 1510b. As illustrated, the retractor 1510 includes two retractor clips 1510b. The retractor clips 1510b interface or interact with the needle holder 260. In the first position, the retraction spring 268 is maintained in a compressed state between the needle holder 260 and the top 1512. The retractor clips 1510b interact with the needle holder 260 to maintain the retraction spring 268 in a compressed state.

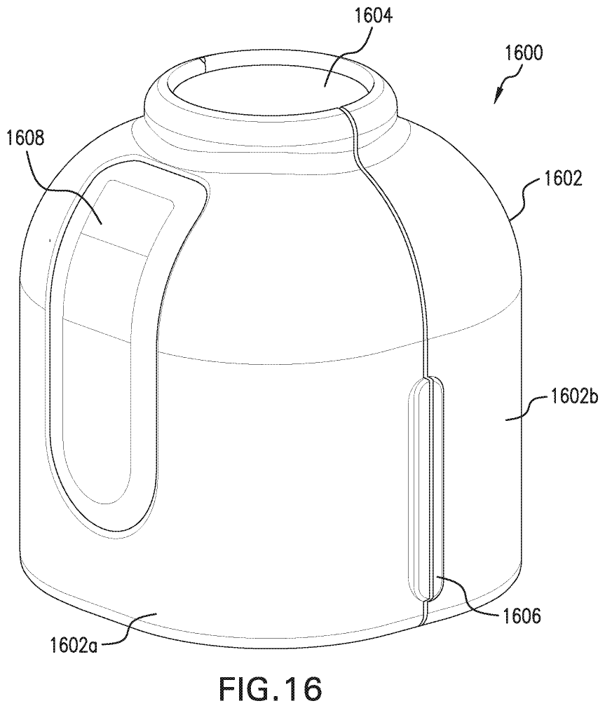

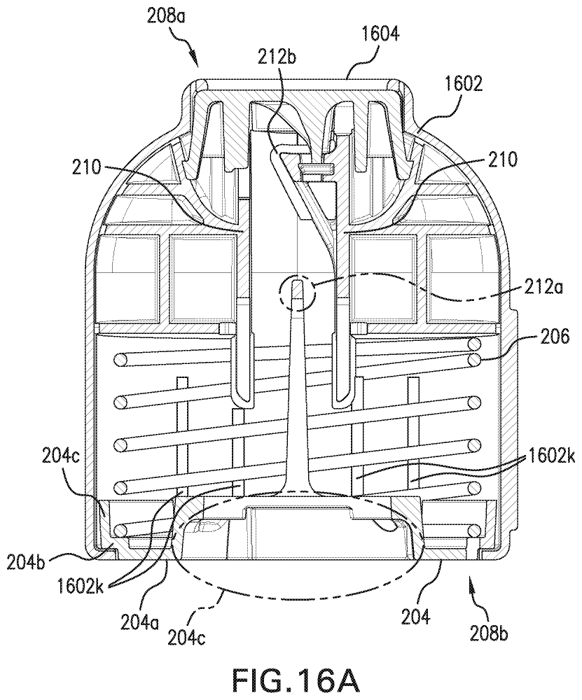

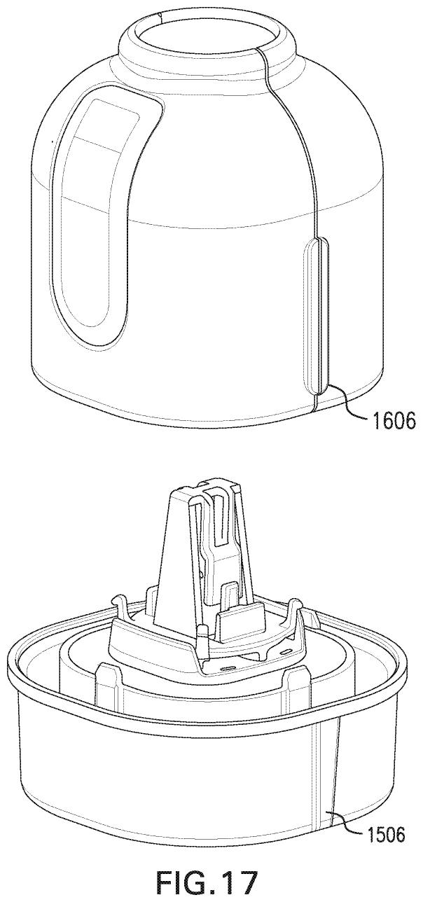

FIG. 16 is an isometric view of an insertion assembly 1600 in a first position, in accordance with embodiments of the present invention. The insertion assembly 1600 includes a housing 1602. In many embodiments, the housing 1602 is made by coupling together two separate pieces such as side A 1602a and side B 1602b. In some embodiments, an alignment feature 1606 is formed when side A 1602a and side B 1602b are coupled together. In other embodiments, the alignment feature 1606 can be found on either side A 1602a or side B 1602b. An actuator 1604, or button, is positioned near the top of the insertion assembly 1600. The actuator 1604 may be retained within the insertion assembly when the side A 1602a and side B 1602b are coupled together. Also included on the insertion assembly 1600 is a side cover 1608. While shown on side A 1602a, the side cover 1608 may alternatively be located on side B 1602b.

FIG. 16A is a cross-section view of the insertion assembly 1600 in a first position, in accordance with embodiments of the present invention. The cross-section view enables details and interactions of internal components of the insertion assembly 1600 to be seen. The insertion assembly 1600 includes a housing 1602 that has a proximal end 208a and a distal end 208b. In the first position, the slider 204 is located within the housing 1602 toward the distal end 208b. The slider 204 further includes a slider cavity 204s that is configured to receive or hold the base of the sensor assembly (not shown). The slider 204 further includes an outer face 204a that is substantially flush with the distal end 208b of the housing 1602. The slider 204 also includes a spring interface 204b that captures an insertion spring 206 between the slider and the housing 1602. The slider 204 additionally includes the tail 212a that interfaces with the latch 212b of the housing 1602.

Within the housing 1602 are the retention walls 210 that extend from the proximal end 208a toward the distal end 208b. The retention walls 210 terminate with a retraction timing 602 within the housing 1602 toward the distal end 208b. The button 1604 is positioned within the housing 1602 and has similar features to the actuator 202 discussed in FIG. 5 that displace the tail 212a of the slider 204 from the latch 212b.

FIG. 17 is an isometric view of positioning of the insertion assembly 1600 in a first position over select components of the sensor assembly 1500 in a first state, in accordance with embodiments of the present invention. FIG. 17 illustrates how the alignment feature 1506 and the alignment feature 1606 can enable both visual and tactile feedback regarding having the insertion assembly 1600 properly aligned with select components of the sensor assembly 1500.

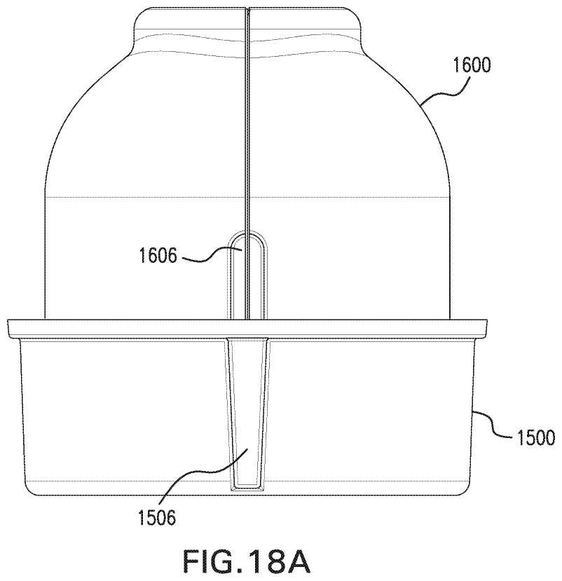

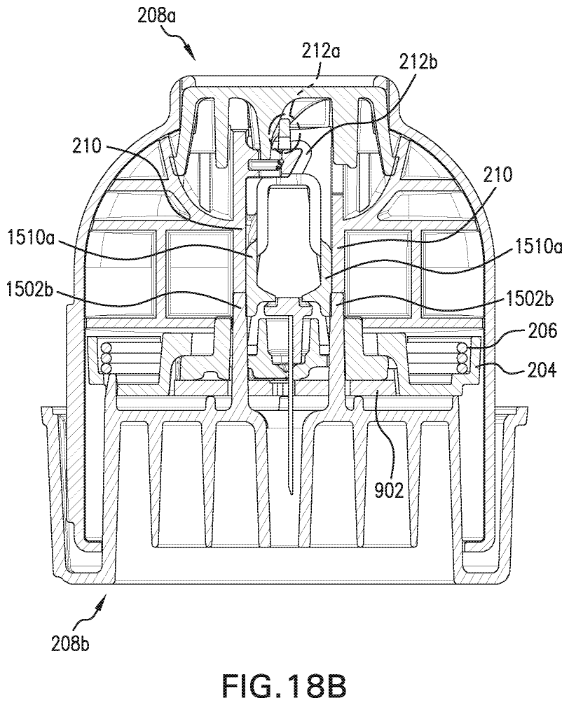

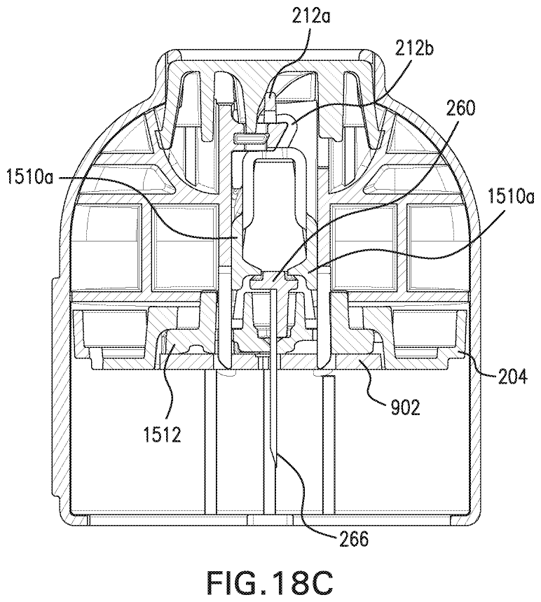

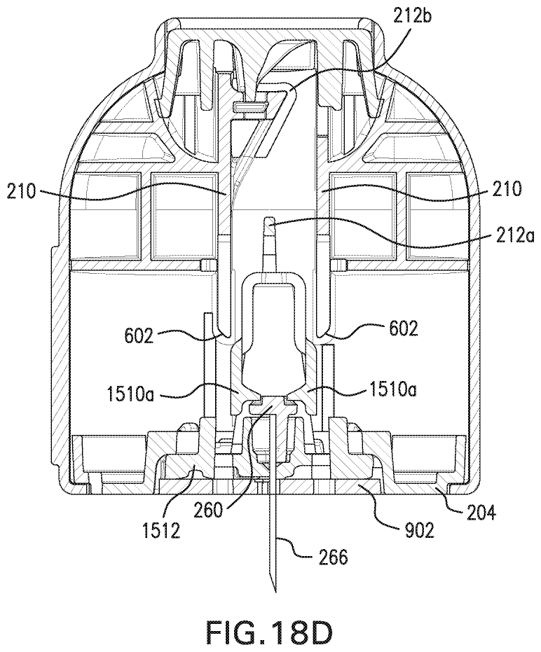

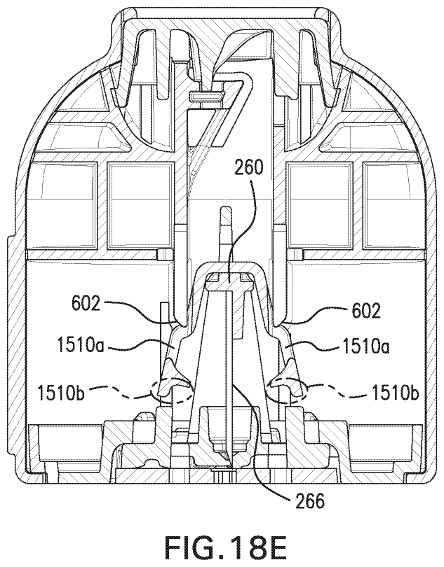

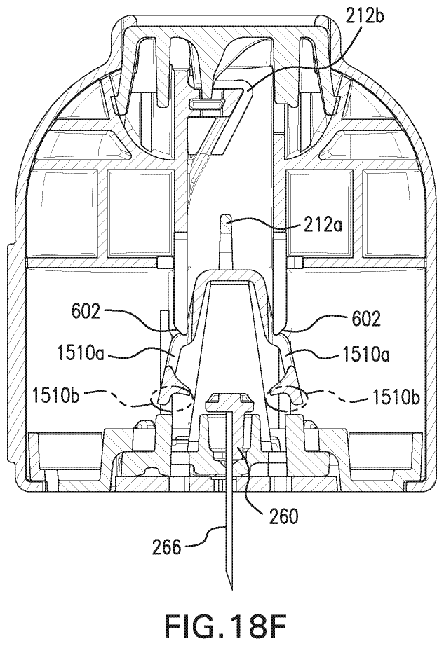

FIG. 18A is a side view of select components of the sensor assembly 1500 in a first state and the insertion assembly 1600 in a second position, in accordance with embodiments of the present invention. This view further illustrates proper alignment between the insertion assembly 1600 and the sensor assembly 1500 via the alignment feature 1606 and the alignment feature 1506. As illustrated, and will be discussed below, the insertion assembly 1600 is in a second position. In the second position, the insertion spring 206 has been compressed and the tail 212a has been secured over the latch 212b. Additionally, the slider 204 has been displaced from the first position to the second position and the clip 1512a of the sensor assembly has been detachably coupled to the slider 204.

FIG. 18B is a cross-section view of select components of the sensor assembly 1500 in a first state and the insertion assembly 1600 in a second position, in accordance with embodiments of the present invention. In the second position, vertical displacement of the insertion assembly 1600 over the stand 1502 results in the vertical displacement of the slider 204 toward the proximal end 208a. Displacement of the slider 204 compresses the insertion spring 206 between the slider 204 and the housing 1602. Additionally, the vertical displacement of the slider 204 results in the tail 212a of the slider 204 being secured over the latch 212b. Other interactions to note that occur between sensor assembly components and insertion assembly components include the interaction between the retention walls 210 and the retractor arms 1510a. While the sensor assembly 1500 has remained in the first state, when it is positioned within the insertion assembly 1600 in a second position, both the posts 1502b of the stand and the retention arms 1510a are positioned to prevent or minimize displacement of the retractor arms 1510a.

FIG. 18C is a cross-section view of select components of the sensor assembly 1500 in a second state and the insertion assembly 1600 in a second position, in accordance with embodiments of the present invention. In the second state the stand 1502 has been separated from the sensor assembly 1500. Removal of the stand 1502 results in removal of the posts 1502b and places the retention walls 210 in contact with the retractor arms 1510a.

FIG. 18D is a cross-section view of select components of the sensor assembly 1500 before a transition between the second state and a third state and the insertion assembly 1600 in an intermediate position between the second position and the first position, in accordance with embodiments of the present invention. In FIG. 18D decompression of the insertion spring (not shown) is displacing the slider 204 toward returning to the first position. As illustrated, the transitional state between the second and third state of the sensor assembly has the retraction timings 602 still in contact with the retractor arms 1510a. Because movement of the retractor arms 1510a is restricted by the retraction timings 602 of the retention walls 210, the retractor clips 1510b remain in contact with the needle holder 260. Accordingly, the retraction spring remains compressed between the needle holder 260 and the top 1512.

FIG. 18E is a cross-section view of select components of the sensor assembly 1500 in a fourth state and the insertion assembly 1600 in a first position, in accordance with embodiments of the present invention. In FIG. 18E the needle holder 260 and needle 266 have been retracted by decompression of the retraction spring vertically displacing the needle holder 260 and needle 266. Upon the retractor arms 1510a clearing the retractor timings 602, the retractor arms 1510a are no longer held in compression by the retractor timings 602. Unrestrained by the retractor timings 602, the retractor arms 1510a decompress or spring open to a decompressed state. In the fourth state, with the needle holder 260 and needle 266 being retracted, the retraction spring (not shown) remains slightly compressed in order to exert a compressive force that prevents the needle holder 260 and needle 266 from being displaced toward the distal end.

FIG. 18F is a cross-section view of select components of the sensor assembly 1500 between the third state and the fourth state and the insertion assembly 1600 in a first position, in accordance with embodiments of the present invention. In FIG. 18F, the retractor arms 1510a have opened to an uncompressed state after clearing the retractor timings 602. As illustrated, the retractor clips 1510b have been displaced from the needle holder 260 but in the intermediary state, the retraction spring has not decompressed and displaced or retracted the needle 266 and needle holder 260.

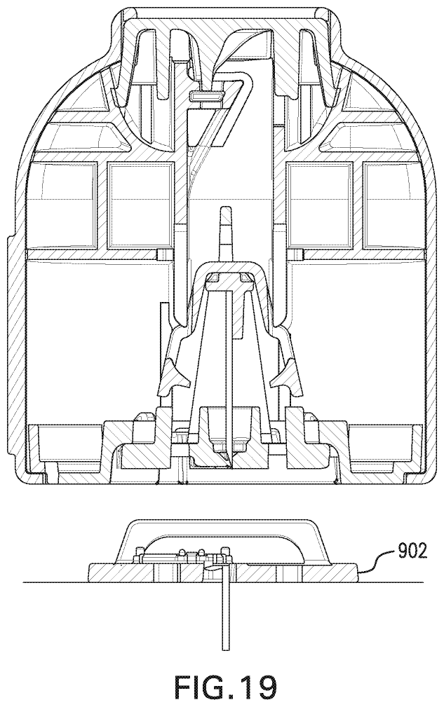

FIG. 19 is a cross-section view of select components of the insertion assembly and the sensor assembly 1500 in a fourth state that includes separation of the base 902, in accordance with embodiments of the present invention. In FIG. 19, a cross-section of the base 902 is shown coupled to a subject and the insertion assembly 1600 having been lifted away from the base 902.

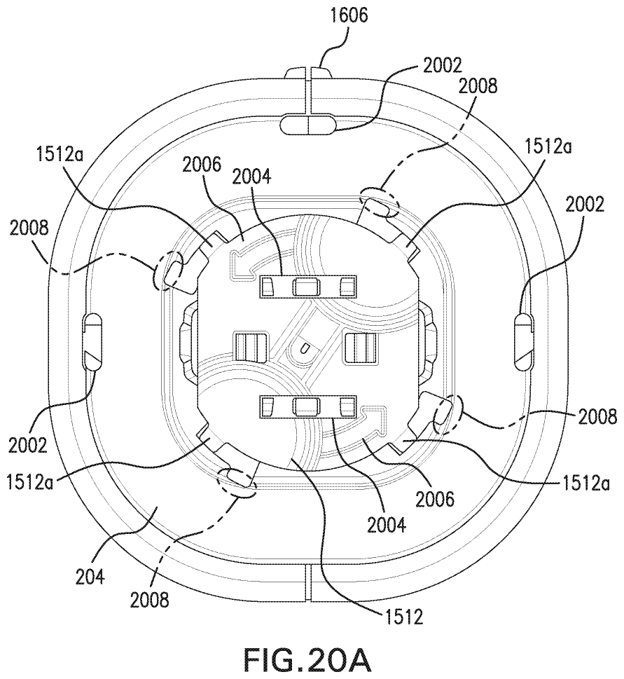

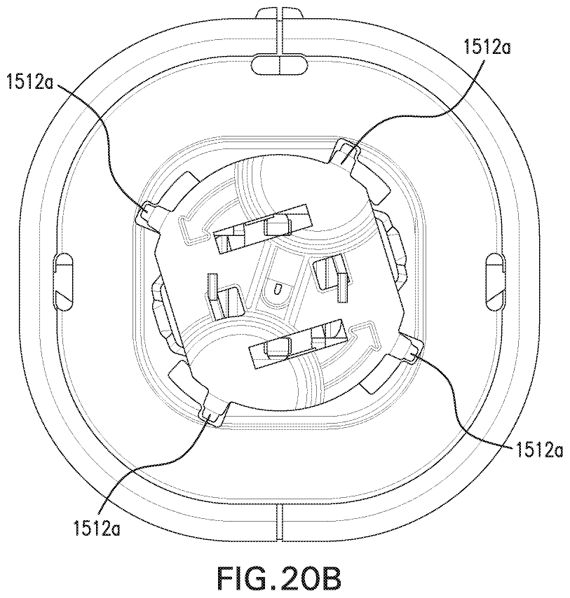

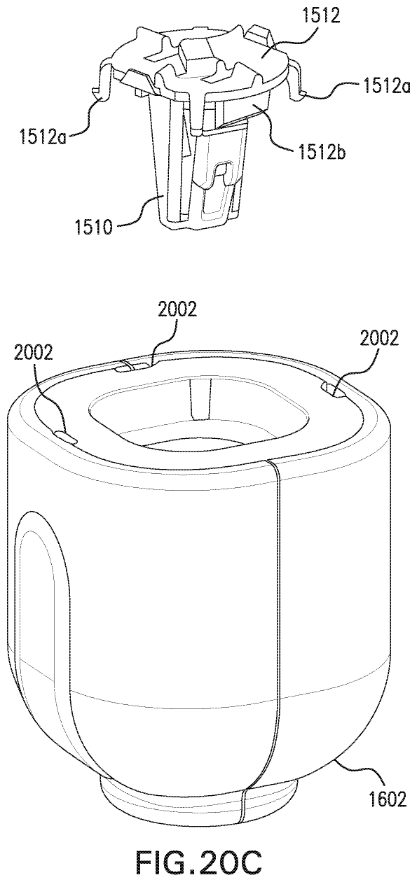

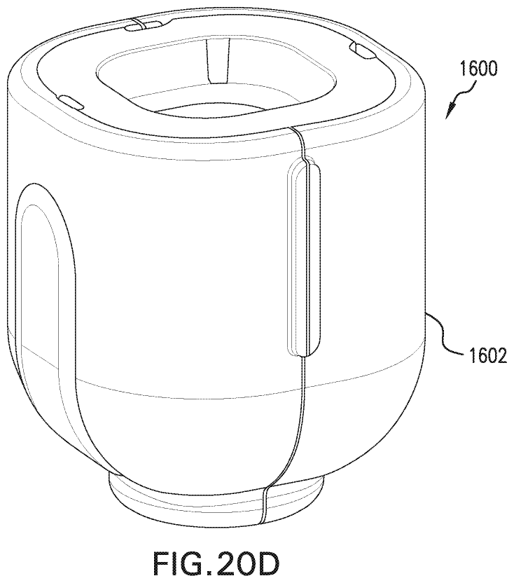

FIG. 20A is a view of the bottom of select components of the sensor assembly 1500 and the insertion assembly 1600 illustrating interactions between the top 1512 and the slider 204, in accordance with embodiments of the present invention. The alignment feature 1606 is visible on an exterior of the insertion assembly 1600. The slider outer face 204a is also visible, along with slider key features 2002 that correspond to key features 1502a, 1502b, and 1502c on the stand 1502 (not shown). Retained within the slider 204 is the top 1512. From this perspective, bases or bottoms of the top clips 1512a are visible along with top apertures 2004. The top apertures 2004 enable the post 1502b of the stand 1502 to pass through the top 1512. The top 1512 further includes indicators 2006 that indicate a direction of twist to remove the top 1512 from the slider 204. When the top 1512 is twisted in the direction of indicators 2006 the clips 1512a can become aligned with slider apertures 2008. When the clips 1512a are aligned with the slider apertures 2008, the top 1512 can be detached from the slider 204.

FIG. 20B is a view of the bottom of select components of the sensor assembly 1500 and the insertion assembly 1600 illustrating removal of sensor assembly 1500 from the insertion assembly 1600, in accordance with embodiments of the present invention. In FIG. 20B, the top 1512 has been rotated in the direction of indicators 2006 to enable removal of the top 1512 from the slider 204. As illustrated, the clips 1512a are visible through the slider apertures 2008.

FIG. 20C is an isometric view illustrating separation of select components of the sensor assembly 1600 from the insertion assembly 1600, in accordance with embodiments of the present invention. Because the top 1512 and retractor 1510 are coupled together, removal of the top 1512 from the insertion assembly 1600 also extracts the retractor 1510. Retained within the retractor 1510 is the needle and needle holder, along with the retraction spring.

FIG. 20D is an isometric view illustrating the insertion assembly 1600 in the first position after removal of the remainder of the sensor assembly 1500, in accordance with embodiments of the present invention. Having returned to the first position and after removal of the remainder of the sensor assembly, the insertion assembly 1600 may be reused with a new sensor assembly 1500 (as illustrated in FIG. 15).

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim requires more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It should be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. An on-body assembly comprising:
an electronics module having a top case and a bottom case;
a base having a clip to retain the electronics module, the base further having an interior base, the interior base including a sensor platform, a sensor channel being formed between the sensor platform and a base aperture, and a retention tab being located over the sensor channel, and
a sensor having a proximal end, a mid-portion and a distal end, the proximal end being coupled to the sensor platform, the mid-portion being positioned within the sensor channel, and the distal end extending through the base aperture.

2. The on-body assembly of claim 1, wherein the base further includes:
a bias face forming a base of the sensor channel, the bias face being positioned below the interior base and below the retention tab.

3. The on-body assembly of claim 2, wherein the bias face is not parallel with the interior base.

4. The on-body assembly of claim 3, wherein the bias face is separated from a retention face of the retention tab by a retention gap.

5. The on-body assembly of claim 3, wherein the mid-portion of the sensor is constrained within the retention gap, the bias face imparting a twist to the mid-portion of the sensor.

6. The on-body assembly of claim 5, wherein the twist to the mid-portion of the sensor biases the distal end in a preferred direction before it passes through the base aperture.

7. The on-body assembly of claim 2, wherein the sensor platform includes a plurality of locating features to position the proximal end of the sensor.

8. The on-body assembly of claim 2, wherein the bottom case of the electronics module includes an interface aperture and a flange, the interface aperture extending between an exterior of the bottom case to the interior of the bottom case.

9. The on-body assembly of claim 8, wherein the clip of the base attaches onto the flange of the bottom case to removably couple the base to the electronics module.

10. The on-body assembly of claim 9, wherein a conductor extends through the interface aperture to make an electrical connection between the electronics module and the proximal end of the sensor.

11. A medical device assembly comprising:
an insertion assembly, and
a sensor assembly that includes a sensor, a base being located on a stand, a top being located on the base, and a retractor being located over the top, the retractor further including at least one retractor arm being in a compressed state, the retractor arm further having a retractor clip that retains a sharp, sharp holder and retraction spring in a compressed state between the retractor clip and the top, wherein the base further includes an interior base, the interior base including a sensor platform, a sensor channel, and at least one retention tab being located over the sensor channel, the sensor channel being formed between the sensor platform and a base aperture,
wherein the sensor has a proximal end, a mid-portion and a distal end, the proximal end being coupled to the sensor platform, the mid-portion being positioned within the sensor channel, and the distal end extending through the base aperture.

12. The medical device assembly of claim 11, wherein the base further includes a bias face, the bias face being positioned at a base of the sensor channel and passing under the at least one retention tab.

13. The medical device assembly of claim 12, wherein the bias face is not parallel to the interior base.

14. The medical device assembly of claim 13, wherein the at least one retention tab includes a retention face being substantially parallel to the bias face.

15. The medical device assembly of claim 14, wherein a retention gap is defined between the retention face and the bias face.

16. The medical device assembly of claim 15, wherein locating the mid-portion of the sensor within the retention gap biases the distal end of the sensor in a preferred direction.

17. The medical device assembly of claim 16, wherein the stand includes at least one post being positioned adjacent to the at least one retractor arm to prevent the retractor arm from transitioning to a decompressed state.

18. The medical device assembly of claim 17, wherein when the at least one retractor arm is in an uncompressed state, the retractor clip release the retraction spring from a compressed state enabling the sharp holder to transition to a retracted position.

19. The medical device assembly of claim 16, wherein the insertion assembly further includes:
an insertion housing having an interior;
a slider being located within the interior of the insertion housing, the slider being movable between a first and second position, the slider including a slider cavity to removable couple the top of the sensor assembly to the slider;
an insertion spring being captured between the slider and the insertion housing.

20. The medical device assembly of claim 18, wherein the insertion housing further includes at least one retention wall formed within the interior of the insertion housing, the retention wall being terminated within the insertion housing with a retraction timing feature.
